SEQUENCE LISTING (10 sequences)

SEQ ID NO: 1 - DNA, 22 bp, artificial
  synthetic oligonucleotide primer
gaccratcctgtcacctctgac

SEQ ID NO: 2 - DNA, 24 bp, artificial
  synthetic oligonucleotide primer
agggcattytggacaaakcgtcta

SEQ ID NO: 3 - DNA, 24 bp, artificial
  synthetic oligonucleotide probe
tgcagtcctcgctcactgggcacg

SEQ ID NO: 4 - DNA, 20 bp, artificial
  synthetic oligonucleotide primer
tgaagacatcgtggcaagag

SEQ ID NO: 5 - DNA, 20 bp, artificial
  synthetic oligonucleotide primer
aggcttgaggtctctgtgga

SEQ ID NO: 6 - DNA, 20 bp, artificial
  synthetic oligonucleotide primer
aagtgtgacacccctggaag

SEQ ID NO: 7 - DNA, 20 bp, artificial
  synthetic oligonucleotide primer
aggtcttggtgtttgccatc

SEQ ID NO: 8 - DNA, 20 bp, artificial
  synthetic oligonucleotide primer
aactttggcattgtggaagg

SEQ ID NO: 9 - DNA, 20 bp, artificial
  synthetic oligonucleotide primer
cacattgggggtaggaacac

SEQ ID NO: 10 - protein, 13 aa, artificial
  autocamtide-2-related inhibitory peptide
KKALRRQEAVDAL

(19) United States
(12) Patent Application Publication
(10) Pub. No.: US 2014/0121237 A1
(43) Pub. Date: May 1, 2014

(54) Methods for Inhibiting Virus Replication

(71) Applicant: UNIVERSITY OF GEORGIA RESEARCH FOUNDATION, INC., ATHENS, GA (US)

(72) Inventors: RALPH A. TRIPP, WATKINSVILLE, GA (US); STEPHEN M. TOMPKINS, WATKINSVILLE, GA (US)

(21) Appl. No.: 14/052,998

(22) Filed: Oct. 14, 2013

Related U.S. Application Data
(63) Continuation-in-part of application No. PCT/US2012/033631, filed on Apr. 13, 2012.
(60) Provisional application No. 61/475,812, filed on Apr. 15, 2011.

(51) Int. Cl. A61K 31/195; A61K 31/18; A61K 45/06; A61K 31/122

(52) U.S. Cl. CPC A61K 31/195; A61K 31/122; A61K 31/18; A61K 45/06
USPC 514/301; 514/562

(57) ABSTRACT

The present invention provides methods for treating a subject having, or at risk of having, a viral infection. The methods include, but are not limited to, the use of 4-(dipropylsulfamoyl)benzoic acid, an inhibitor of CDC25B phosphatase, an inhibitor of ICAM-1, an inhibitor of CamK2B, or a combination thereof.

### CROSS-REFERENCE TO RELATED APPLICATIONS

This application is a continuation-in-part of PCT/US2012/033631, filed Apr. 13, 2012, published in the English language on Oct. 18, 2012 as International Publication No. WO 2012/142492 A2, which claims the benefit of U.S. Provisional Application Ser. No. 61/475,812, filed Apr. 15, 2011, each of which is incorporated by reference herein.

### GOVERNMENT FUNDING

This invention was made with government support under HHSN266200700006C awarded by the National Institute of Allergy and Infectious Disease. The government has certain rights in the invention.

### BACKGROUND

Influenza A viruses continue to cause yearly epidemics and periodic pandemics in humans. Recent estimates are that 20% of the world population is afflicted every season (Girard et al., 2010, Vaccine 28:4895-902). Influenza viruses are members of the Orthomyxoviridae family having single stranded negative sense segmented RNA. The segmented nature of the genome and error-prone viral RNA polymerase provide a strong source of change and mutations that confer immune escape. Influenza viruses undergo natural mutation over time, or antigenic drift, a feature requiring the need for new vaccines to be developed annually to confer protection against heterovariant strains. Periodically, influenza viruses simultaneously infect a “mixing vessel”, e.g swine, leading to viruses with new gene segments and an antigenic shift that may cause a pandemic. Although influenza vaccines are generally safe and effective, they cannot always meet the population coverage demands, are less efficacious in the populations at greatest risk (e.g. persons <5 or >65 years of age) (Fiore et al., 2007, MMWR Recomm Rep 56:1-54) and due to the short time frame between identification of a pandemic strain and need for vaccination, are not always available or efficacious.

An option to control infection in influenza afflicted or at-risk people is the use of antiviral drugs. Current FDA-approved antiviral drugs are M2-ion channel inhibitors, i.e. adamantanes, and neuraminidase inhibitors (NAI), e,g, zanamivir and oseltamivir (McKimm-Breschkin, 2005, Treat Respir Med 4:107-16, Monto, 2003, Vaccine 21:1796-800, Pinto et al., 1992, Cell 69:517-28). Adamantanes work by blocking infection at an early stage of virus replication (Wang et al., 1993, J Virol 67:5585-94), while NAI block virus progeny release at the later stage of virus replication (Gubareva et al., 2000, Lancet 355:827-35). Despite the utility of these antiviral drugs, new and novel antivirals are being sought due to the emergence of drug resistance (Boltz et al., 2010, Drugs 70:1349-62, Deyde et al., 2007, J Infect Dis 196:249-57, Layne et al., 2009, Science 323:1560-1, Nguyen et al., 2010, PLoS One 5:e9332, Simonsen et al., 1997, Am J Public Health 87:1944-50). There are several reports of new influenza therapies, particularly those targeting host genes required for influenza virus replication (Brass et al., 2009, Cell 139:1243-54, Bushman et al., 2009, PLoS Pathog 5:e1000437, Hao et al., 2008, Nature 454:890-3, Karlas et al., 2010, Nature 463:818-22, Konig et al., 2009, Nature 463:813-7, Min and Subbarao, 2010, Nat Biotechnol 28:239-40, Watanabe et al., 2010, Cell Host Microbe 7:427-39). Targeting host genes offers an innovative and generally refractory approach to drug resistance because influenza virus requires many host pathways during their life cycle (Konig et al., 2009, Nature 463:813-7, Watanabe et al., 2010, Cell Host Microbe 7:427-39), and host gene targets are typically stable.

### SUMMARY OF THE INVENTION

The present invention includes methods for treating a viral infection using antiviral drugs that do not require binding to a viral antigen that is susceptible to antigenic drift. The present invention provides methods for treating a subject. In one embodiment, the method includes administering to a subject an effective amount of a composition that includes 4-(dipropylsulfamoyl)benzoic acid, wherein the subject has, or is at risk of having, an influenza virus infection, wherein the subject has no greater than a detectable level of oseltamivir carboxylate. In one embodiment, the method includes administering to a subject an effective amount of a composition that includes 4-(dipropylsulfamoyl)benzoic acid, wherein the subject has, or is at risk of having, a virus infection, wherein the virus infection is not an influenza virus infection. In one embodiment, the method includes administering to a subject an effective amount of a composition that includes an inhibitor of CDC25B phosphatase, wherein the subject has, or is at risk of having, a virus infection. In one embodiment, the method includes administering to a subject an effective amount of a composition that includes an inhibitor of ICAM-1, wherein the subject has, or is at risk of having, a virus infection.

In one embodiment, one or more hydrogen-bearing carbon atoms in the 4-(dipropylsulfamoyl)benzoic acid may be substituted, wherein each substituent is selected from a halogen, a nitrile, a hydroxy, an alkoxy (OR), a nitrate, a nitrite, a sulfate (O—SO₃R), an amino (NR₂), a nitro, a sulfonate (SO₂OR), or a C1-C10 organic group, wherein each R is independently a hydrogen or an organic group.

In one embodiment, the inhibitor of CDC25B phosphatase is selected from NSC95397, NSC115447, NSC135880, NSC139049, or NSC672121. In one embodiment, one or more hydrogen-bearing carbon atoms in the NSC95397, NSC115447, NSC135880, NSC139049, or NSC672121 may be substituted, wherein each substituent is selected from a halogen, a nitrile, a hydroxy, an alkoxy (OR), a nitrate, a nitrite, a sulfate (O—SO₃R), an amino (NR₂), a nitro, a sulfonate (SO₂OR), or a C1-C10 organic group, wherein each R is independently a hydrogen or an organic group.

In one embodiment, the inhibitor of ICAM-1 is 4-[(4-Methyl phenyl)thio]thieno[2,3-c]pyridine-2-carboxamide. In one embodiment, one or more hydrogen-bearing carbon atoms in the 4-[(4-Methyl phenyl)thio]thieno[2,3-c]pyridine-2-carboxamide is substituted, wherein each substituent is selected from a halogen, a nitrile, a hydroxy, an alkoxy (OR), a nitrate, a nitrite, a sulfate (O—SO₃R), an amino (NR₂), a nitro, a sulfonate (SO₂OR), or a C1-C10 organic group, wherein each R is independently a hydrogen or an organic group.

A method of the present invention may further include administering to the subject a composition that includes 4-(dipropylsulfamoyl)benzoic acid, an inhibitor of CDC25B phosphatase, an inhibitor of CamK2B, an inhibitor of ICAM-1, or a combination thereof.

In one embodiment, the inhibitor of CamK2B is KN-62, KN-93, arcyriaflavin A or SEQ ID NO:10. In one embodiment, one or more hydrogen-bearing carbon atoms in the KN-62, KN-93, or arcyriaflavin A may be substituted, wherein each substituent is selected from a halogen, a nitrile, a hydroxy, an alkoxy (OR), a nitrate, a nitrite, a sulfate (O—SO₃R), an amino (NR₂), a nitro, a sulfonate (SO₂OR), or a C1-C10 organic group, wherein each R is independently a hydrogen or an organic group.

In one embodiment, the virus infection is a virus infection of the respiratory tract. The virus infection may include an influenza virus, such as influenza virus A, influenza virus B, or a combination thereof.

The present invention also includes uses of an inhibitor of CDC25B phosphatase and/or an inhibitor of ICAM-1 in the preparation of a medicament for a viral infection, and uses of an inhibitor of CDC25B phosphatase and/or an inhibitor of ICAM-1 for treating a viral infection.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Inhibition of CAMKIIB prevents A/WSN/33 replication in a dose-dependent manner in BEAS2B cells. (A) Treatment with 100-250 uM KN-93 has no detectable cell cytotoxicity as the concentrations examined; however treatment with 500 uM modestly but significantly (p<0.05) affected cell viability compared to control (diluent); (B) qPCR was used to detect virus M gene copies; all treatments are significantly different than control (*p=0.000983, **p=0.000828***p=0.003919 Student's t-test). The experiment shown is representative of three independent experiments performed.

FIG. 2. CDC25B inhibitor prevents A/WSN/33 replication in a dose-dependent manner in BEAS2B cells. (A) NP-staining of BEAS2B cells transfected with RISC-free control, siRNA targeting MEK, or siRNA targeting CDC2B; (B) Western blot of level of CDC25B gene expression after siRNA targeting in BEAS2B cells; (C) qPCR detection of virus copies 24 hpi in BEAS2B cell treated with 0.24-7.8 uM NSC 95397; (D) percent BEAS2B cell viability 24 h post-drug treatment; *p=0.0002, **p=0.005, ***p=0.003; Student's t-test). The experiment shown is representative of three independent experiments performed.

FIG. 3. CDC25B and CaMKIIB kinase inhibitors do not substantially affect virus entry and infection. Analysis of influenza virus nuclear protein staining as an indicator of A/WSN/33 virus infection of BEAS2B cells after control or inhibitor treatment. Photographic (A) representation of NP staining, and quantitative (B) analysis of the number of NP stained plaques in the lawn of control and KN-93 treated (250 uM) or NSC 95397-treated BEAS2B cells. The photographic representation shown is representative of four independent experiments performed.

FIG. 4. Combination of CDC25B and CaMKIIB inhibitors has an additive effect in preventing A/WSN/33 influenza viral replication. Prophylactic treatment of BEAS2B with 250 uM KN-93, 1.95-7.8 uM NSC 95397, or combination of 250 uM KN-93 plus 1.95-7.8 uM NSC 95397 significantly inhibited influenza M gene copy number as determined by qPCR; all treatments are significantly different than control (*p=0.000983, **p=0.000828***p=0.003919 Student's t-test). The experiment shown is representative of three independent experiments performed.

FIG. 5. Prophylactic treatment with NSC 95397 or KN-93 inhibits A/WSN/33 replication. Female BALB/c mice were prophylactically treated with diluent control, 10 mg/kg KN-93, or 2.5 mg/kg NSC 95397 for 24 (A) or 48 hours (B) before infection with A/WSN/33. Forty-eight hours after infection, the lungs were harvested for RNA and qPCR performed to determine M gene copy. (A) Control treated versus NSC 95397 versus KN-93 group; all treatments significantly (p<0.05) different than control; (B) control versus NSC95397, p<0.05; control versus KN-93 was not statistically significant. N=5 mice/group/experiment; the experiment shown is representative of three independent experiments performed.

FIG. 6. Prophylactic treatment with NSC 95397 or KN-93 inhibits A/CA/04/09 replication. Female BALB/c mice were prophylactically treated with diluent control, 10 mg/kg KN-93, or 2.5 mg/kg NSC 95397 for 24 (A) or 48 hours (B) before infection with A/CA/04/09. Forty-eight hours after infection, the lungs were harvested and infectious titers determined as TCID₅₀ on MDCK cells. (A) Control treated versus NSC 95397 versus KN-93 group; all treatments significantly (p<0.05) different than control; (B) control versus NSC95397, p<0.05; control versus KN-93 was not statistically significant. Lung virus titers in drug treated mice were significantly (p<0.05) different compared to controls; N=5 mice/group/experiment.

FIG. 7. (A) RNA interference of SLC22A8 gene expression in A549 cells inhibits influenza A virus (A/WSN/33) replication as determined by NP staining 48 after infection with a MOI=1.0; (B) SLC22A8 gene expression in A549 cells treated with varying concentrations of Probenicid at 24 and 48 h post-treatment as determined by qRT-PCR.

FIG. 8. Fold SLC22A8 gene expression in A549 cells infected with A/WSN/33 following treatment with varying concentrations of Probenicid at 24 and 48 h post-treatment as determined by qRT-PCR.

FIG. 9. Copy number of A/WSN/33 in A549 cells treated with varying concentrations of Probenecid at 24 and 48 hpi.

FIG. 10. Evaluation of A/WSN/33 infection determined by monoclonal antibody staining of A/WSN/33 nucleoprotein (NP) expressed in A549 cells at 24 and 48 h post-infection following treatment with varying doses of Probenecid.

FIG. 11. Determining the inhibitory concentration −50 (IC₅₀) of Probenecid at 24, 36, and 48 h post-A/WSN/33 infection of A549 cells.

FIG. 12. Evaluating different concentrations of Probenecid for efficacy against a different strain of influenza virus, A/New Calcdonia/20/90, at 24 and 48 h post-infection of A549 cells.

FIG. 13. (A) Evaluating efficacy of various concentrations of Probencid to inhibit SLC22A8 gene expression in the lungs of mice at 24 and 48 h post-treatment; (B) Evaluating efficacy of various concentrations of Probencid to inhibit A/WSN/33 replication in the lungs of mice as determined by qRT-PCR copy number at 24 and 48 g post-treatment.

FIG. 14. (A) Evaluating efficacy of 10 or 200 mg/kg of Probencid to inhibit A/New Calcdonia/20/90 replication in the lungs of mice at 24 h post-treatment; (B) Evaluating efficacy of 10 or 200 mg/kg of Probencid to inhibit A/New Calcdonia/20/90 replication in the lungs of mice at 48 h post-treatment.

FIG. 15. (A & B). Individual experiments each evaluating the efficacy of 10 mg/kg Probencid prophylaxis 1 day prior to virus infection (−24), or treatment one day after virus infection (+24) to inhibit A/WSN/33 replication in the lungs of mice in the presence of Tamiflu (Pb+Tamilflu), or efficacy of Tamiflu alone.

FIG. 16. CDC25B is a pro-influenza A host factor. To evaluate the role of CDC25B during IAV infection, A549 cells were transfected with non-targeting siRNA (siNEG) or siRNA targeting CDC25B (siCDC25B). At 48 hours post-transfection, cells were infected with influenza A/WSN/33 at MOI=0.05 and fixed (A) or harvested (B) for RNA isolation at 48 hpi. A) Following fixation, cells were stained for influenza virus NP and nuclei (DAPI). Cells were visualized using high-content imaging system. Three representative images for each condition were shown (top) and %-NP positive cells were quantified from ten fields (bottom). B) Culture supernatants from infected cells were used for virus titration in MDCK cells. C) Knock-down of CDC25B mRNA expression was verified using qRT-PCR normalized to GAPDH. *p<0.05; **p<0.01.

FIG. 17. NSC95397 resulted in increased levels of phosphorylated CDK1 and ERK1/2. A) BEAS2B cells were treated with DMSO (0 μM) or increasing doses of NSC95397 (1, 5, or 10 μM). At 1 hour post-treatment, cells were mock-infected or infected with influenza A/WSN/33 at MOI=1. Cells were harvested for protein analyses at 7 hpi. B) Cells were DMSO-treated or treated with 5 μM NSC95397. At 1 hour post-treatment, cells were mock-infected or infected with influenza A/WSN/33 at MOI=1. Cells were harvested for protein analyses at 1, 3, or 7 hpi. To determine effects of NSC95397 in dephosphorylation of CDC25B targets CDK1 and ERK1, protein lysates were subjected to immunoblot using phospho-specific and total CDK1 and ERK1/2 antibodies. Levels of cellular CDC25B, GAPDH, and viral NP proteins were also evaluated.

FIG. 18. CDC25B inhibitor NSC95397 limits influenza A and B infections in vitro. BEAS2B cells were infected with influenza A/WSN/33 (A, B) or B/Florida/04/06 (C, D) at MOI=0.05 in presence of increasing NSC95397 concentration (in ¼-log increment). At 24 hpi, cell supernatants were harvested for virus titration in MDCK cells. Dotted lines indicate 50% inhibition of virus titer (IC₅₀). E) BEAS2B cells were treated with increasing dose of NSC95397 (in ½-log increment) and cellular cytotoxicity was assessed at 24 hours post-treatment using ToxiLight bioassay kit. Percent cell viability of NSC95397-treated cells was determined relative to non-treated (100% viability) and lysed cells (0% viability). Dotted line indicates 50% cytotoxicity (CC₅₀). D) IC₅₀ and CC₅₀ values were determined using non-linear regression method. Selectivity indices were calculated as the ratio of CC₅₀ to IC₅₀.

FIG. 19. NSC95397 reduces abundance of viral RNA and increased IFNβ expression. BEAS2B cells were infected with influenza A/WSN/33 at MOI=1. A, B) To assess when NSC95397 acts to block IAV infection, 2 μM NSC95397 was added at different time points pre- or post-infection. C) To determine if abundance of specific viral RNA species were reduced in presence of NSC95397, total RNA were isolated at 7 hpi and subjected to qRT-PCR analysis using primers specific to cRNA, mRNA, and vRNA of IAV segment 5. Viral RNA abundance was normalized to GAPDH and their abundance in NSC95397 relative to DMSO-treated cells were graphed. D) Total RNA were isolated at 4 or 7 hpi and subjected to IFNβ qRT-PCR. IFNβ mRNA abundance was normalized to GAPDH and its abundance in DMSO- or NSC95397-treated cells relative to non-infected/non-treated BEAS2B cells was graphed. n.s.: not significant; *p<0.05; **p<0.01; ***p<0.001.

FIG. 20. NSC95397 anti-influenza action is mediated by modulation ofes viral NS1 nuclear localization and RNA binding function in the nucleus. (A-C) BEAS2B were infected with influenza A/WSN/33 at MOI=1 in presence of DMSO or NSC95397 and harvested or fixed at 4 or 7 hpi. A) To evaluate IRF-3 phosphorylation following infection, protein lysates extracted at 4 or 7 hpi were used for immunoblot analyses using antibodies against phosphorylated (P-IRF-3), total IRF-3, viral NS1, and GAPDH as loading control. B) Fixed cells were stained for NS1 (red), NP (green), and nuclei (DAPI; blue). White arrows signify nuclear NS1 staining C) Protein extracts from 7 hpi were subjected to subcellular fractionation. Total cell lysate and chromatin-bound protein fraction were used for immunoblot analyses using antibodies against viral NS1 and NP proteins, and cellular GAPDH, histone H3, and transcription factor SP 1 as fractionation controls. At 7 hpi, viral NS1 and NP can be found associated with cellular chromatin in DMSO-treated, but not in NSC95397-treated cells. D) BEAS2B cells were infected with wild type (wt) or reconstructed influenza A/WSN/33 virus expressing NS1 R38AK41A (NS1 RK) in presence of DMSO or 2 μM NSC95397 at MOI=0.05. Cells were collected for virus titration at 24 hpi. n.s.: not significant; ***p<0.001.

FIG. 21. NSC95397 limits pathogenesis of A/WSN/33 infection in vivo. BALB/c female mice were infected with (A-B) lethal dose (10³ PFU) or (C) sub-lethal dose (70 PFU) of A/WSN/33 and treated with DMSO or 2.5 mg/kg NSC95397 orally at 24 hour pre- or post-infection. Mice survival (A) and weight loss (B) were monitored daily for 14-days according to guidelines from the IACUC of the University of Georgia. C) RNA was isolated from lungs of infected mice at 72 hpi and virus copy numbers were evaluated by qRT-PCR. *p<0.05; **p<0.01; ***p<0.001.

### DETAILED DESCRIPTION OF ILLUSTRATIVE EMBODIMENTS

The present invention provides methods for using compounds to inhibit a virus. In one embodiment, a virus may be a member of the family Orthomyxoviridae, for instance, a member of the genus Influenzavirus A, a member of the genus Influenzavirus B, a member of the genus Influenzavirus C, or a member of the genus Thogotovirus. Type species that are members of the genus Influenzavirus A, include, but are not limited to, Influenza A virus. Serotypes of the type species Influenza virus A include, but are not limited to, H1N1, H1N2, H2N2, H3N1, H3N2, H3N8, H5N1, H5N2, H5N3, H5N8, H5N9, H7N1, H7N2, H7N3, H7N4, H7N7, H9N2, and H10N7. The skilled person will recognize that other serotypes are possible in view of antigenic drift and the simultaneous infection of one animal with different influenza viruses. Type species that are members of the genus Influenzavirus B include, but are not limited to, Influenza B virus. Type species that are members of the genus Influenzavirus C include, but are not limited to, Influenza C virus. Type species that are members of the genus Thogotovirus include, but are not limited to, Thogoto virus and Dori virus. Serotypes of the type species Dhori virus include, but are not limited to, Batken virus and Dhori virus. In one embodiment, a virus may be Respiratory syncytial virus, human metapneumovirus, corona virus (e.g. SARS), Parainfluenza virus, Hepatitis A, Hepatitis B, Hepatitis C, measles virus, and mumps virus.

Compounds useful in the methods described herein inhibit virus replication. In one embodiment, a compound inhibits replication of an influenza virus, such as an Influenza A virus or an Influenza B virus. A suitable in vitro assay for determining whether a compound inhibits viral replication is shown in Example 1. Briefly, a compound that is to be tested for the ability to inhibit virus replication is serially diluted in a medium suitable for use with cultured cells. The cells that are to be infected with the virus are pretreated with the different concentrations of the compound, and then incubated for a period of time, such as an hour, before infection. Suitable cells include cultured primary epithelial cells obtained from the respiratory tract of an individual, or an appropriate cultured cell line, such as BEAS2B (ATCC CRL-9609) or A546 (ATCC CCL-185). The virus may be added to the cells at a suitable multiplicity of infection to result in detectable viral replication in control cells not exposed to the compound. After 24 hours incubation, virus may be detected using any suitable method, such as qRT-PCR. In one embodiment, a compound is considered to inhibit virus replication if there is a statistically significant decrease in the amount of replication compared to virus replication in control cells not exposed to the compound. In one embodiment, a compound is considered to inhibit virus replication if there is a decrease in the amount of replication of at least 10%, at least 20%, at least 30%, at least 40%, at least 50%, at least 60%, at least 70%, or at least 80% compared to a control not contacted with the compound.

A suitable in vivo assay for determining whether a compound inhibits viral replication is shown in Example 1. Briefly, an appropriate animal, such as a mouse is exposed to a virus at a concentration sufficient to cause in infection. The route of administration of the virus varies depending upon the virus. In the case of an Influenza virus, mice may be anesthetized and intranasally administered a suitable amount of virus, for instance, 10⁵ pfu. Routes for administration vary depending upon the virus used and are known to the skilled person. The compound may be administered to the animal before exposure or after exposure to the virus. Timing of administration of the compound can vary, and may be at 24 hours or 48 hours before exposure to the virus, and may be at 24 hours or 48 hours after exposure to the virus. At varying times after exposure to the virus, an appropriate tissue is removed to determine the concentration of virus. When the virus is an Influenza virus, the tissue may be lung. The tissue may be homogenized and then standard methods for quantifying virus number may be used, such as plaque assay, 50% tissue culture infective does (TCID50), hemagglutination assay, or qPCR. In one embodiment, a compound is considered to inhibit virus replication if there is a statistically significant decrease in the amount of replication compared to virus replication in a control animal not exposed to the compound. In one embodiment, a compound is considered to inhibit virus replication if there is a decrease in the amount of replication of at least 10%, at least 20%, at least 30%, at least 40%, at least 50%, at least 60%, at least 70%, or at least 80% compared to a control animal not contacted with the compound.

In one embodiment, a compound useful in the methods described herein is 4-(dipropylsulfamoyl)benzoic acid, which has the following structure,

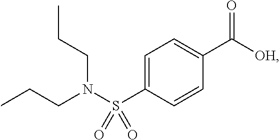 [SMILES: CCCN(CCC)S(=O)(=O)c1ccc(C(=O)O)cc1]

and is also known by the generic name Probenecid. Other examples of compounds useful in the methods described herein include the 2-nitro, 2-hydroxyl, and 2-chloro analogs of Probenecid (Blanchard et al., 1972, J. Pharmacol. Exp. Therapeutics, 180(2):397-410, see also Sheikh et al., 1979, Biochem. Pharmacol., 28:15-22). The skilled person will recognize that the hydrogen atom on one or more of the hydrogen-bearing carbon atoms in the molecule can be substituted with a substituent including, but not limited to, halogen (e.g., F, Cl, Br, I), nitrile (CN), hydroxy (OH), alkoxy (OR), nitrate (O—NO₂), nitrite (O—N═O), sulfate (O—SO₃R), amino (NR₂), nitro (NO₂), sulfonate (SO₂OR), or a C1-C10 organic group (e.g., in some embodiments a C1-C4 organic group or moiety), with each R independently being hydrogen or an organic group. Whether an analog of Probenecid is useful in the methods described herein can be determined by a suitable in vitro assay for viral replication, or by a suitable in vivo assay for viral replication.

In one embodiment, a compound useful in the methods described herein inhibits CDC25B phosphatase. Examples of CDC25B phosphatase inhibitors include, but are not limited to, compounds disclosed by Lazo et al. (2002, Mol. Pharmacol., 61:720-728);

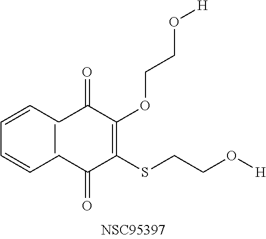 [SMILES: O=C1C(OCCO)=C(SCCO)C(=O)c2ccccc21]

(and a fluorinated form thereof, see Park et al., 2007, Bioorganic Medicinal Chem. Lett., 17(8):2351-2354),

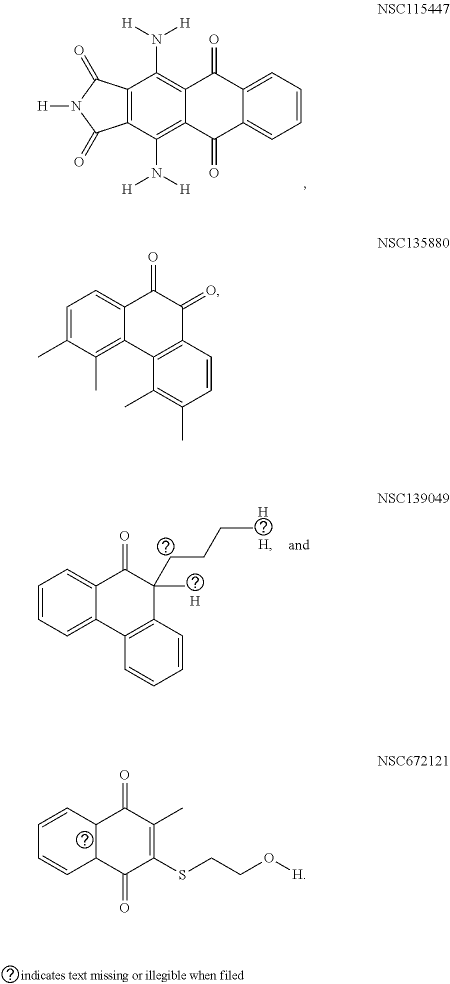

Other examples of CDC25B phosphatase inhibitors disclosed in Lazo et al. include NSC4056, NSC5069, NSC50554, NSC92217, NSC102381, NSC102539, NSC105326, NSC107043, NSC117271, NSC125248, NSC128981, NSC129077, NSC129141, NSC130442, NSC135879, NSC327705, NSC668394, and. Other examples of CDC25B phosphatase inhibitors include, but are not limited to, compounds disclosed in Lazo and Wipf (2008, Anticancer Agents Med Chem., 8(8):837-842): dysidiolide, dnacin A1, sulfurcin, SC-ααδ9, Taiho acid, seco-cholestane, Pharmacia-Upjohn tetrahydroisoquinoline, menadione, DA3003-1 (NSC663284), indolyldihydroxyqinone, BN82002, fascaplysin, PM-20, xenicane diterpenoid, and Ugi peptide mimic. The skilled person will recognize that the hydrogen atom on one or more of the hydrogen-bearing carbon atoms in the molecule can be substituted with a substituent including, but not limited to, halogen (e.g., F, Cl, Br, I), nitrile (CN), hydroxy (OH), alkoxy (OR), nitrate (O—NO₂), nitrite (O—N═O), sulfate (O—SO₃R), amino (NR₂), nitro (NO₂), sulfonate (SO₂OR), or a C1-C10 organic group (e.g., in some embodiments a C1-C4 organic group or moiety), with each R independently being hydrogen or an organic group. Whether a compound that inhibits CDC25B phosphatase is useful in the methods described herein can be determined by a suitable in vitro assay for viral replication, or by a suitable in vivo assay for viral replication.

In one embodiment, a compound useful in the methods described herein inhibits Ca-Calmodulin kinase 2b (Camk2b). Examples of such compounds include, but are not limited to,

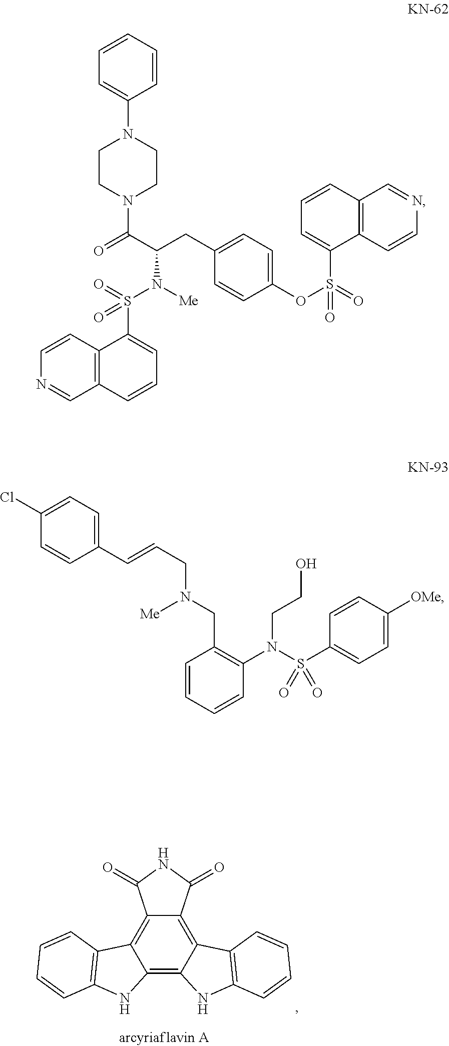 [SMILES: CN([C@@H](Cc1ccc(OS(=O)(=O)c2cccc3cnccc23)cc1)C(=O)N1CCN(c2ccccc2)CC1)S(=O)(=O)c1cccc2cnccc12 ; COc1ccc(S(=O)(=O)N(CCO)c2ccccc2CN(C)C/C=C/c2ccc(Cl)cc2)cc1 ; O=C1NC(=O)c2c1c1c3ccccc3[nH]c1c1[nH]c3ccccc3c21]

and autocamtide-2-related inhibitory peptide KKALRRQEAVDAL (SEQ ID NO:10). The skilled person will recognize that the hydrogen atom on one or more of the hydrogen-bearing carbon atoms in the molecule can be substituted with a substituent including, but not limited to, halogen (e.g., F, Cl, Br, I), nitrile (CN), hydroxy (OH), alkoxy (OR), nitrate (O—NO₂), nitrite (O—N═O), sulfate (O—SO₃R), amino (NR₂), nitro (NO₂), sulfonate (SO₂OR), or a C1-C10 organic group (e.g., in some embodiments a C1-C4 organic group or moiety), with each R independently being hydrogen or an organic group. Whether a compound that inhibits Camk2b is useful in the methods described herein can be determined by a suitable in vitro assay for viral replication, or by a suitable in vivo assay for viral replication.

In one embodiment, a compound useful in the methods described herein inhibits ICAM-1. An example of such compounds includes, but is not limited to, 4-[(4-Methyl phenyl)thio]thieno[2,3-c]pyridine-2-carboxamide:

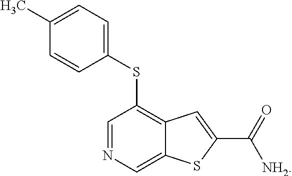 [SMILES: Cc1ccc(Sc2cncc3sc(C(N)=O)cc23)cc1]

The skilled person will recognize that the hydrogen atom on one or more of the hydrogen-bearing carbon atoms in the molecule can be substituted with a substituent including, but not limited to, halogen (e.g., F, Cl, Br, I), nitrile (CN), hydroxy (OH), alkoxy (OR), nitrate (O—NO₂), nitrite (O—N═O), sulfate (O—SO₃R), amino (NR₂), nitro (NO₂), sulfonate (SO₂OR), or a C1-C10 organic group (e.g., in some embodiments a C1-C4 organic group or moiety), with each R independently being hydrogen or an organic group. Whether a compound that inhibits ICAM-1 is useful in the methods described herein can be determined by a suitable in vitro assay for viral replication, or by a suitable in vivo assay for viral replication.

As used herein, the term “organic group” is used for the purpose of this invention to mean a hydrocarbon group that is classified as an aliphatic group, cyclic group, or combination of aliphatic and cyclic groups (e.g., alkaryl and aralkyl groups). In the context of the present invention, suitable organic groups for compounds of this invention are those that do not interfere with the ability of a compound to inhibit virus replication. In the context of the present invention, the term “aliphatic group” means a saturated or unsaturated linear or branched hydrocarbon group. This term is used to encompass alkyl, alkenyl, and alkynyl groups, for example. The term “alkyl group” means a saturated linear or branched monovalent hydrocarbon group including, for example, methyl, ethyl, n-propyl, isopropyl, tert-butyl, amyl, heptyl, and the like. The term “alkenyl group” means an unsaturated, linear or branched monovalent hydrocarbon group with one or more olefinically unsaturated groups (i.e., carbon-carbon double bonds), such as a vinyl group. The term “alkynyl group” means an unsaturated, linear or branched monovalent hydrocarbon group with one or more carbon-carbon triple bonds. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” or “aryl group” means a mono- or polynuclear aromatic hydrocarbon group. The term “heterocyclic group” means a closed ring hydrocarbon in which one or more of the atoms in the ring is an element other than carbon (e.g., nitrogen, oxygen, sulfur, etc.).

As a means of simplifying the discussion and the recitation of certain terminology used herein, the terms “group” and “moiety” are used to differentiate between chemical species that allow for substitution or that may be substituted and those that do not so allow for substitution or may not be so substituted. Thus, when the term “group” is used to describe a chemical substituent, the described chemical material includes the unsubstituted group and that group with nonperoxidic O, N, S, Si, or F atoms, for example, in the chain as well as carbonyl groups or other conventional substituents. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, tert-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, sulfoalkyls, etc. On the other hand, the phrase “alkyl moiety” is limited to the inclusion of only pure open chain saturated hydrocarbon alkyl substituents, such as methyl, ethyl, propyl, tert-butyl, and the like.

The present invention is also directed to compositions including one or more of the compounds described herein. Such compositions typically include a pharmaceutically acceptable carrier. As used herein “pharmaceutically acceptable carrier” includes, but is not limited to, saline, solvents, dispersion media, coatings, antibacterial and antifungal agents, isotonic and absorption delaying agents, and the like, compatible with pharmaceutical administration. Additional active compounds can also be incorporated into the compositions.

A composition may be prepared by methods well known in the art of pharmacy. In general, a composition can be formulated to be compatible with its intended route of administration. A formulation may be solid or liquid. Administration may be systemic or local. In some aspects local administration may have advantages for site-specific, targeted disease management. Local therapies may provide high, clinically effective concentrations directly to the treatment site, with less likelihood of causing systemic side effects.

Examples of routes of administration include parenteral (e.g., intravenous, intradermal, subcutaneous, intraperitoneal, intramuscular), enteral (e.g., oral), and topical (e.g., epicutaneous, inhalational, transmucosal) administration. Appropriate dosage forms for enteral administration of a compound described herein may include tablets, capsules or liquids. In one embodiment, Probenecid is in the form of a tablet for oral administration. Appropriate dosage forms for parenteral administration may include intravenous administration. Appropriate dosage forms for topical administration may include nasal sprays, metered dose inhalers, dry-powder inhalers or by nebulization.

Solutions or suspensions can include the following components: a sterile diluent such as water for administration, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; electrolytes, such as sodium ion, chloride ion, potassium ion, calcium ion, and magnesium ion, and agents for the adjustment of tonicity such as sodium chloride or dextrose. pH can be adjusted with acids or bases, such as hydrochloric acid or sodium hydroxide. A composition can be enclosed in ampoules, disposable syringes or multiple dose vials made of glass or plastic.

Compositions can include sterile aqueous solutions or dispersions and sterile powders for the extemporaneous preparation of sterile solutions or dispersions. For intravenous administration, suitable carriers include physiological saline, bacteriostatic water, Cremophor EL™ (BASF, Parsippany, N.J.) or phosphate buffered saline. A composition is typically sterile and, when suitable for injectable use, should be fluid to the extent that easy syringability exists. It should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile solutions can be prepared by incorporating the active compound (e.g., a compound described herein) in the required amount in an appropriate solvent with one or a combination of ingredients such as those enumerated above, as required, followed by filtered sterilization. Generally, dispersions are prepared by incorporating the active compound into a sterile vehicle, which contains a dispersion medium and other ingredients such as from those enumerated above. In the case of sterile powders for the preparation of sterile injectable solutions, methods of preparation that may be used include vacuum drying and freeze-drying which yields a powder of the active ingredient plus any additional desired ingredient from a previously sterile-filtered solution thereof.

Oral compositions may include an inert diluent or an edible carrier. For the purpose of oral therapeutic administration, the active compound can be incorporated with excipients and used in the form of tablets, troches, or capsules. Oral compositions can also be prepared using a fluid carrier. Pharmaceutically compatible binding agents can be included as part of the composition. The tablets, pills, capsules, troches and the like may contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the active compounds may be delivered in the form of an aerosol spray, a nebulizer, or an inhaler, such as a nasal spray, metered dose inhaler, or dry-powder inhaler.

Systemic administration can also be transmucosal or transdermal. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds may be formulated into ointments, salves, gels, or creams as generally known in the art. An example of transdermal administration includes iontophoretic delivery to the dermis or to other relevant tissues.

The active compounds may be prepared with carriers that will protect the compound against rapid elimination from the body, such as a controlled release formulation, including implants. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially. Liposomal suspensions can also be used as pharmaceutically acceptable carriers. These can be prepared according to methods known to those skilled in the art. Delivery reagents such as lipids, cationic lipids, phospholipids, liposomes, and microencapsulation may also be used.

In one embodiment, an active compound may be associated with a targeting group. As used herein, a “targeting group” refers to a chemical species that interacts, either directly or indirectly, with the surface of a cell, for instance with a molecule present on the surface of a cell, e.g., a receptor. The interaction can be, for instance, an ionic bond, a hydrogen bond, a Van der Waals force, or a combination thereof. Examples of targeting groups include, for instance, saccharides, polypeptides (including hormones), polynucleotides, fatty acids, and catecholamines. Another example of a targeting group is an antibody. The interaction between the targeting group and a molecule present on the surface of a cell, e.g., a receptor, may result in the uptake of the targeting group and associated active compound.

Toxicity and therapeutic efficacy of such active compounds can be determined by standard pharmaceutical procedures in cell cultures or experimental animals, e.g., for determining the ED₅₀ (the dose therapeutically effective in 50% of the population).

The data obtained from cell culture assays and/or animal studies can be used in formulating a range of dosage for use in humans or other animals. The dosage of such active compounds lies preferably within a range of concentrations that include the ED₅₀ with little or no toxicity. The dosage may vary within this range depending upon the dosage form employed and the route of administration utilized. For an active compound used in the methods of the invention, it may be possible to estimate the therapeutically effective dose initially from cell culture assays. A dose may be formulated in animal models to achieve a concentration range that includes the IC₅₀ (i.e., the concentration of the test compound which achieves a half-maximal inhibition of signs and/or symptoms) as determined in cell culture. Such information can be used to more accurately determine useful doses in humans or other animals.

The compositions can be administered one or more times per day to one or more times per week. The skilled artisan will appreciate that certain factors may influence the dosage and timing required to effectively treat a subject, including but not limited to the severity of the infection, previous treatments, the general health and/or age of the subject, and other diseases present. Moreover, treatment of a subject with an effective amount of a compound can include a single treatment or can include a series of treatments.

The present invention includes methods for using the compounds disclosed herein. In one embodiment, a method includes contacting a cell with an effective amount of a compound. In one embodiment, the contacting is under conditions suitable for allowing the compound to interact with the surface of the cell. In one embodiment, the contacting is under conditions suitable for introduction of a compound into the cell.

Conditions that are “suitable” for an event to occur, such as introduction of a compound into a cell, or “suitable” conditions are conditions that do not prevent such events from occurring. Thus, these conditions permit, enhance, facilitate, and/or are conducive to the event. As used herein, an “effective amount” relates to a sufficient amount of a compound to provide the desired effect. For instance, in one embodiment an “effective amount” is an amount effective to decrease viral replication in a cell. In one embodiment, a cell is considered to have a decrease in viral replication if there is a decrease of at least 10%, at least 20%, at least 30%, at least 40%, at least 50%, at least 60%, at least 70%, or at least 80% compared to a control not contacted with the compound. In one embodiment an “effective amount” is an amount effective to decrease the production of infectious virus particles. In one embodiment, a cell is considered to have a decrease in the production of infectious virus particles if there is a decrease of at least 10%, at least 20%, at least 30%, at least 40%, at least 50%, at least 60%, at least 70%, or at least 80% compared to a control not contacted with the compound.

A cell that may be used in the methods described herein may be ex vivo or in vivo. As used herein, “ex vivo” refers to a cell that has been removed from the body of an animal. Ex vivo cells include, for instance, primary cells (e.g., cells that have recently been removed from a subject and are capable of limited growth in tissue culture medium), and cultured cells (e.g., cells that are capable of long term culture in tissue culture medium). Examples of primary cells include cells normally present in an animal's respiratory tract, including, but not limited to, epithelial cells, endothelial cells, and cells from tissues such as, but not limited to, spleen, lymph node, liver, blood, kidney, bone marrow, and central nervous system. Useful cultured cells include those used for viral replication. Examples of cultured cells include, but are not limited to, epithelial cells, such as BEAS2B (ATCC CRL-9609) or A546 (ATCC CCL-185), and endothelial cells. Control cells may be obtained from the ATCC and may be cultured according to methods known in the art. Control cells may also be obtained from tissue samples through, for example, biopsy. As used herein, “in vivo” refers to a cell that is present within an animal. A cell that may be used in the methods described herein may be an avian cell from an avian species, or a mammalian cell, such as, for instance, a mouse cell, a rat cell, a primate cell (e.g., monkey, human), a rabbit cell, a gerbil cell, a guinea pig cell, a ferret cell, or a cell from a swine species.

The present invention also includes methods for treating a viral infection. In one embodiment, a method includes treating a viral infection in a subject, where a subject in need thereof is administered an effective amount of a composition that includes a compound described herein. The subject may be a mammal, such as a member of the family Muridae (a murine animal such as rat or mouse), a primate, (e.g., monkey, human), a gerbil, a guinea pig, a ferret, or a swine species, or the subject may be an avian species. As used herein, the term “infection” refers to the invasion of a host's cells by a virus and subsequent replication within the cell. An infection typically results in a reaction by the host, where such a reaction is any deviation from or interruption of the normal structure or function of a part, organ, or system, or combination thereof, of a subject, that is manifested by a characteristic symptom or clinical sign.

As used herein, the term “symptom” refers to subjective evidence of an infection experienced by a patient and caused by the infection. As used herein, the term “clinical sign,” or simply “sign,” refers to objective evidence of an infection present in a subject. Symptoms and/or signs associated with infections referred to herein and the evaluation of such signs are routine and known in the art. Examples of signs of a viral infection vary depending upon the type of virus causing an infection in the subject, and can be readily evaluated by the skilled person. A symptom and/or sign may be localized, systemic, or a combination thereof. Whether a subject has an infection, and whether a subject is responding to treatment, may be determined by evaluation of signs associated with the infection.

Viral infections include viral infections of the respiratory tract. Examples of viruses causing infections of the respiratory tract include, but are not limited to, influenza viruses, such as influenza virus A, influenza virus B, influenza virus C, Respiratory syncytial virus, human metapneumovirus, corona virus (e.g. SARS), measles virus, and Parainfluenza virus. Viral infections include viral infections of the liver. Examples of viruses causing infections of the liver include, but are not limited to, Hepatitis A, Hepatitis B, and Hepatitis C.

In one embodiment, a viral infection treated using a method of the present invention is flu, which is caused by an influenza virus. Signs of flu caused by an influenza virus may include chills, fever, sore throat, muscle pains, severe headache, coughing, weakness/fatigue, general discomfort, nausea, vomiting, and/or signs of pneumonia, such as chest pain, and difficulty breathing.

Treatment of a viral infection can be prophylactic or, alternatively, can be initiated after the development of a viral infection. Treatment that is prophylactic, for instance, initiated before a subject manifests signs of a viral infection, is referred to herein as treatment of a subject that is “at risk” of developing a viral infection. An example of a subject that is at risk of developing a viral infection is a person having a risk factor. Treatment can be performed before, during, or after the occurrence of the viral infections described herein. Treatment initiated after the development of a viral infection may result in decreasing the severity of the signs of the infection, or completely removing the signs. An “effective amount” may be an amount effective to alleviate one or more symptoms and/or signs of the infection. In one embodiment, an effective amount is an amount that is sufficient to effect a reduction in a symptom and/or sign associated with a viral infection. A reduction in a symptom and/or a sign is, for instance, at least 10%, at least 20%, at least 30%, at least 40%, at least 50%, at least 60%, at least 70%, at least 80%, at least 90%, or at least 100% in a measured sign as compared to a control, a non-treated subject, or the subject prior to administration of the compound. It will be understood, however, that the total daily usage of the compounds described herein will be decided by an attending physician within the scope of sound medical judgment. The exact amount required will vary depending on factors such as the type of disease being treated.

The compounds described herein may also be administered to a subject in combination with other therapeutic agents to increase the overall therapeutic effect. Therapeutic agents useful for the treatment of the viral infections described herein are known and used routinely. In one embodiment, a therapeutic agent may include Oseltamivir (available under the trade designation TAMIFLU, from Genentech, South San Francisco, Calif.). Oseltamivir is converted to oseltamivir carboxylate after administration to a subject. In one embodiment, when Probenecid is used to treat a viral infection caused by an influenza virus, the subject does not include a detectable level of oseltamivir carboxylate, or does not include any oseltamivir carboxylate.

The present invention is illustrated by the following examples. It is to be understood that the particular examples, materials, amounts, and procedures are to be interpreted broadly in accordance with the scope and spirit of the invention as set forth herein.

### Example 1

Influenza A viruses continue to have a major health impact worldwide affecting people of all ages, but often have the greatest effect on the elderly, young children, and those vulnerable with underlying conditions. The most effective means for controlling infection is vaccination; however, there is a need for anti-viral drug options. Influenza M2 inhibitor and neuraminidase inhibitor drugs are available but drug resistance has emerged and spread. Research efforts have recently focused on identification of host genes that are essential for influenza replication as they offer potential targets for drug discovery and are refractory to the development of resistance. Using RNA interference high-throughput genome-wide assays to identify candidate host genes that are required during influenza replication, cell division cycle 25 (CDC25), a member of the CDC25 family of phosphatases, was identified as an important host factor required for virus replication. This study shows that CDC25B gene expression in human bronchial (BEAS2B) cells can be silenced with NSC95397, an inhibitor of CDC25B phosphatase, to inhibit influenza replication without causing toxicity, suggesting targeting of CDC25B phosphatase is a therapeutic approach to treat influenza infection.

### Materials and Methods

Cells and viruses. A549 (ATCC CCL-185) type II respiratory epithelial cells were maintained in DMEM medium containing 10% FBS (Hyclone, Logan, Utah) and BEAS2B (ATCC CRL-9609) human bronchoepithelial cells were maintained in Bronchial Epithelial Basal medium (BEBM, Lonza, Walkerville, Md., USA) supplemented with 30 ug/ml bovine pituitary extract, 0.5 ug/ml hydrocortisone, 0.5 ng/ml human recombinant epidermal growth factor, 0.5 ug/ml epinephrine, 10 ug/ml transferring, 5 ug/ml insulin, 0.1 ng/ml retinoic acid, and 6.5 ng/ml triiodothyronine (BEGM, SingleQuots, Lonza, Walkerville, Md., USA) at 37° C. in a 5% CO₂ incubator. Influenza A viruses, A/WSN/33 and A/CA/04/09, were propagated in 9 day-old embryonated chicken eggs obtained from a specific-pathogen-free leghorn chicken flock (Sunrise Farms, Catskill, N.Y., USA), the allantoic fluid from this parental stock was tested for hemagglutination (HA) activity. HA positive allantoic fluids were pooled, aliquoted, and stored at −80° C. until use. The virus titer was calculated as a 50% tissue culture infectious dose (TCID₅₀) by endpoint dilution on Madin-Darby canine kidney cells (MDCK) as previously described (Reed and Meunch. 1938, Am. J. 488 Hyg 27:493-497, Smith et al., 2011, Viral immunology 24:131-42). MDCK cells were cultured in OPTI-MEM I (Invitrogen, Carlsbad, Calif., USA).

Reverse transfection. Lyophilized CDC25B and MEK siRNAs (Dharmacon ThermoFisher, Lafayette, Colo.) were diluted with HBSS (HyClone, Logan, Utah) and allowed to incubate for 5 minutes. Dharmafect-1 transfection reagent (Lafayette, Colo.) and HBSS were added such that each well of a 96-well plate (Nunc) received 0.04 ml of transfection reagent and 0.096 ml of HBSS. The siRNA/transfection reagent mix was allowed to incubate for 20 minutes at room temperature after which 0.08 ml of 1.5×10⁴ A549 cells suspended in DMEM/5% FBS was added to each well, and the plate incubated for 48 hours at 37° C. in 5% CO₂. The final concentration of siRNA for all transfections was 50 nM. For the toxicity screen, Toxilight (Lonza, Lonza, Walkerville, Md., USA) reagent was used to visualize cell cytotoxicity 48 h after siRNA transfection. At 36 hours post infection supernatants were harvested and virus titers were determined by TCID50 on MDCK cells. Cell monolayers were analyzed for the presence of influenza nucleoprotein (NP) by immunostaining. The screens were run in triplicate.

In vitro inhibition assays. KN-93 (SIGMA, St. Louis, Mo.) and NSC 95397 (TOCRIS Bioscience, Ellisville, Mich.) were dissolved in DMSO and serially diluted in BEBM media starting from 500 uM and 250 uM, respectively. The BEAS2B cells were washed with PBS once and then pretreated with increasing concentrations of KN-93 or NSC 95607 for 1 hour before infection. Cellular toxicity was determined by the ToxiLight BiAssay kit assay (Lonza, Walkerville, Md., USA). A/WSN/33 was added to the wells (volume of virus=1% of total volume) without changing medium at a multiplicity of infection (MOI) of 0.05. After 24 hours post-infection (pi), supernatant and remaining cells in triplicates were used for isolation mRNA for detection of virus copies by qRT-PCR. For RNA isolation we used the Qiagen RNAeasy kit (Qiagen, Md., USA) and for detection the master mix prepared using specific primers for influenza A virus (INFA-F (SS118272-45) 5′ dGACCRATCCTGTCACCTCTGAC (SEQ ID NO:1) and INFA-R(SS118272-46) 5′ dAGGGCATTYTGGACAAAKCGTCTA 3′ (SEQ ID NO:2) INFA-P (SS118273-01)5′ d FAM TGCAGTCCTCGCTCACTGGGCACG-BHQ-1 (SEQ ID NO:3) (Bioresearch Technologies, Inc., Novato, Calif., provided by CDC). The quantifications were previously calibrated and optimized using One Step RT-PCR kit (Qiagen) with 30 min at 50° C. for the reverse transcription reaction, 15 min at 95° C. for the Taq inhibitor activation, and PCR amplification with 95° C., 15 sec, 55° C. for 30 sec and a total of 45 cycles in an MX3005P thermocycler (Strategene, Agilent Technologies, Santa Clara, Calif.).

Western Blotting. Cultured cells and supernatants were lysed in 1M Tris-HCL, 1M NaCl, 40 mM EDTA, 100 mM NaF, Sodium Deoxycholate and cocktail tablet and protein quantified BCA kit (Pierce, Rockford, Ill., USA). Twenty nanograms of proteins were mixed with SDS-PAGE sample buffer and fractionated in 4-20% SDS-PAGE and transferred to nitrocellulose membranes. The membranes were stained with Ponceau S and then blocked with 1% nonfat milk in 0.15 M NaCl, 10 mM Tris-HCl pH 7.4 containing 0.05% Tween 20 before incubation with one of the following monoclonal or polyclonal antibodies: rabbit polyclonal anti-CDC25B (Ab 63220, ABCAM, Cambridge, Mass.) and mouse monoclonal anti-GAPDH (Ab8245, ABCAM, Cambridge, Mass.). Reactivity was developed after incubation with the respective peroxidase- or avidin-conjugated secondary antibody (mouse or rabbit anti-human IgG), or streptavidin144 peroxidase and ECF (Pierce, Rockford, Ill., USA).

Immunostaining of infected cells. After treatment with drugs and infection with virus, the cells were fixed in cold methanol:acetone (80:20) for 10 min. The fixative was removed, cells washed with PBS and incubated 1 h PBS, 0.1% Tween (PBST) containing 1% BSA, followed by incubation with mouse monoclonal anti-NP (H16-L10, IgG2a, Biovest International Inc., NCCC) in the same buffer. After 1 h, the cells were washed in PBST and incubated with anti IgG-HRP (Invitrogen, Carlsbad, Calif., USA) for 1 hour. Bound antibodies were detected by TMB reagent (Vector, Burlingame, Calif.). The number of positively stained cells for the antibody was counted in 10 fields for each well using the 20× objective and Zeiss microscope.

Animals and infections. BALB/c female mice (6-8 week-old) were obtained from NCI. All experiments and procedures were Institutional Animal Care and Use Committee (IACUC) approved by the IACUC of UGA. Prior to infection, mice were anesthetized with intraperitoneal treatment with 250 mg/kg Avertin, and intranasally infected with 10⁵ pfu of A/WSN/33 or A/CA/04/09 in 50 μl PBS. Body weight and survival were evaluated daily. All experiments were performed with 5 animals per group and repeated independently at least twice.

Antiviral treatments. Mice were administered 10 mg/kg KN-93 or 2.5 mg/kg NSC95397 or PBS as a control, by gavage. Prophylactic treatments were started 24 hours or 48 hours before the infection, and lungs collected at time-points after infection. In the therapeutic approach, infected mice were treated with each compound at 24 or 48 hours after infection.

qRT-PCR and TCID₅₀ of lungs of infected mice. The lungs of infected animals were longitudinally sectioned for qRT-PCR and TCID₅₀. One half of the lung was added to TRizol (Invitrogen, Carlsbad, Calif., USA) and used to isolate total RNA. Complementary DNA (cDNA) was synthesized using 5 ng of RNA through a reverse transcription reaction (Superscript III, Invitrogen). Real time PCR quantitative mRNA analyses were performed for CAMKIIB, CDC25b and GAPDH, which was used for normalization with the Sybergreen kit (Qiagen, Md., USA). The reactions conditions were: 50° C. 2 min for cDNA amplification followed by one cycle of 15 min at 95° C. and, 40 cycles of 30 s at 94° C., 1 min at 55° C. and 30 s at 72° C. The reactions were finished by one cycle of 1 min at 95° C., 30 s at 55° C. and 30 s at 95° C. The following primers were used: CAMKIIB forward 5′-TGAAGACATCGTGGCAAGAG (SEQ ID NO:4) and CAMKIIB reverse 5′-AGGCTTGAGGTCTCTGTGGA (SEQ ID NO:5); CDC25B forward 5′ AAGTGTGACACCCCTGGAAG (SEQ ID NO:6) and CDC25B reverse 5′AGGTCTTGGTGTTTGCCATC (SEQ ID NO:7); GAPDH forward 5′-AACTTTGGCATTGTGGAAGG (SEQ ID NO:8) and GAPDH reverse 5′CACATTGGGGGTAGGAACAC(SEQ ID NO:9). For TCID₅₀ analysis, 1 ml of PBS was added to each lung tissue sample and homogenized using the TissueLyser (Qiagen Valencia, Calif.), then centrifuged at 10,000 rpm for 5 min. The TCID₅₀ was determined for each sample as previously described (37). Briefly, 10-fold serial dilutions of samples from 10⁻¹ to 10⁻⁶ were made in Modified eagles medium (MEM) with TPCK [1-(tosylamido-2-pheyl)ethyl chloromethyl ketone]-treated trypsin (WorthingtonBiochemical Corporation, Lakewood, N.J.) (1 ug/ml). Dilutions of each sample were added to Madin-Darby canine kidney (MDCK; ATCC) cells (4 wells/dilution; 200 ul/well) and the cells were incubated for 48 h at 37° C. The contents of each well were tested for hemagglutination and the TCID₅₀ was calculated by the Reed and Meunch method (Hinshaw et al., 1981, Infection and immunity 34:354-61, Reed and Meunch. 1938, Am. J. 488 Hyg 27:493-497) each experiment was representative of three replicates.

Statistical analyses. The Student's t test was used throughout, except for the survival curves where the One-Way ANOVA and the Mantel-Cox (log-rank) test were employed. P values of <0.05 were considered statistically significant.

### Results

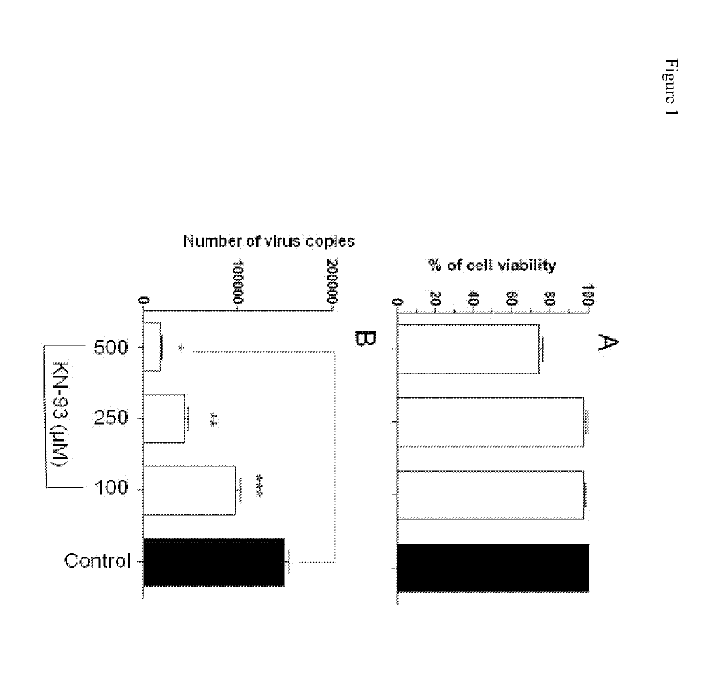

Inhibition of CAMK2B prevents A/WSN/33 replication in BEAS2B cells. It was previously shown that KN-93, a potent inhibitor of CaM kinase II (CAMK2B) (Anderson et al., 1998, J Pharmacol Exp Ther 287:996-1006), inhibits A/WSN/33 influenza viral replication in a dose-dependent manner in MDCK cells (McKimm-Breschkin, 2005, Treat Respir Med 4:107-16). The results from our RNAi human siRNA genome (siGENOME) screen also identified CAMK2B as a critical gene required for influenza virus replication; therefore, we examined the effect of KN-93 on influenza replication in BEAS2B human lung epithelia cells. BEAS2B cells were infected with A/WSN/33 virus (MOI=0.05) in the presence of increasing concentrations of KN-93. Cell toxicity was determined at each concentration of KN-93 (FIG. 1A). At the highest drug concentration (500 uM), low levels (20-30%) of cell cytotoxicity were observed compared to the control. Cell cytotoxicity was negligible at 250 uM, a concentration that inhibited >80% of virus replication (FIG. 1B), and insignificant at lower drug concentrations. As expected, KN-93 treatment inhibited influenza replication in a dose-dependent manner (FIG. 1B). Our findings are consistent with those shown in MDCK cells (McKimm-Breschkin, 2005, Treat Respir Med 4:107-16), indicating that CaMKIIB is required for influenza replication, and that this gene can be targeted in human lung epithelial cells to reduce virus replication.

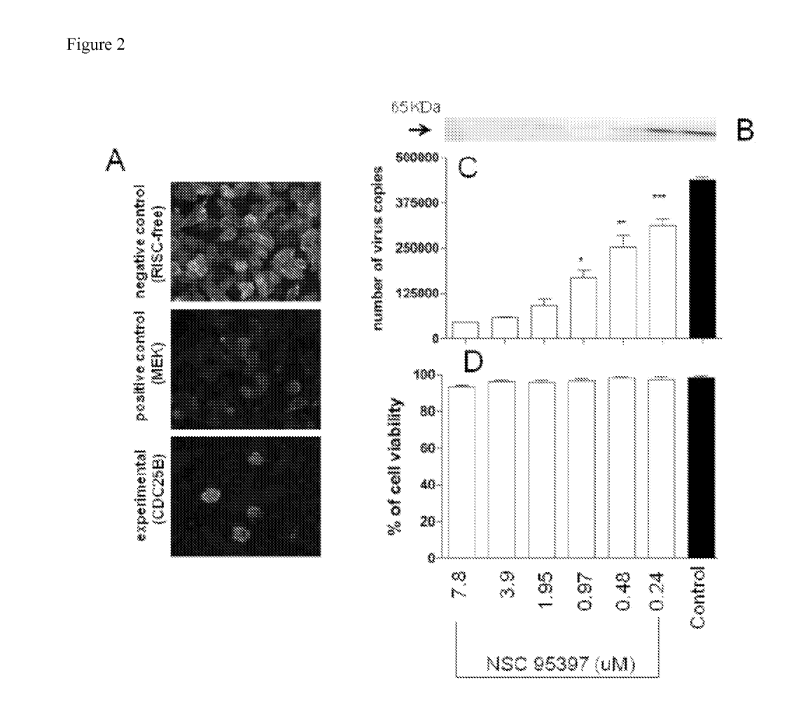

NSC 95397, a CDC25B inhibitor, prevents influenza replication in BEAS2B cells. A second gene identified by RNAi siGENOME screening was cell division cycle 25 (CDC25), which is a member of the CDC25 family of phosphatases (Rudolph. 2007, Biochemistry 46:3595-604), and previously shown to be present in many human tumors (Park et al., 2007, Bioorg Med Chem Lett 17:2351-4). Inhibition of CDC25 phosphatases has been shown to inhibit carcinoma cell growth and block G2/M phase transition in cultured cells (Lazo et al., 2002, Mol Pharmacol 61:720-8). To determine if CDC25B is a druggable target for inhibiting influenza virus, siRNA targeting this gene, and selective and irreversible inhibitors of CDC25B phosphatase, NSC 95397, were evaluated in BEAS2B cells (FIG. 2). The effect of siRNA silencing CDC25B on A/WSN/33 replication was determined by influenza nucleoprotein (NP) staining of BEAS2B cell (FIG. 2A). Compared to controls, cells depleted of MEK (Pleschka et al., 2001, Nature cell biology 3:301-5) or CDC25B show a remarkable decrease in NP staining at 48 hours post infection (FIG. 2A). To evaluate the CDC25B inhibitor, NSC 95397, BEAS2B cells were treated for 1 h with NSC 95397, or solvent control, and then infected (MOI 0.05) with A/WSN/33 virus. NSC 95397 treatment inhibited CDK1 phosphorylation (Boutros et al., 2006. Current opinion in cell biology 18:185-91), and decreased the amount of CDC25B protein as shown by Western blot (65 KDa band) using rabbit polyclonal anti-CDC25B antibody (FIG. 2B). BEAS2B cells prophylactically treated with NSC 95397 had significantly reduced levels of virus copies at 24 hpi compared to control treated cells as determined by qRT-PCR (FIG. 2C). NSC 95397 prevented A/WSN/33 influenza virus replication in BEAS2B cells in a dose-dependent manner, and virus copies were significantly inhibited at concentrations as low as 0.24 uM (IC₅₀=0.9 uM). Importantly, no substantial cell cytotoxicity was evident at concentrations above 7.8 μM (FIG. 2D). These results indicate that CDC25B phosphatase has an important role in influenza virus replication.

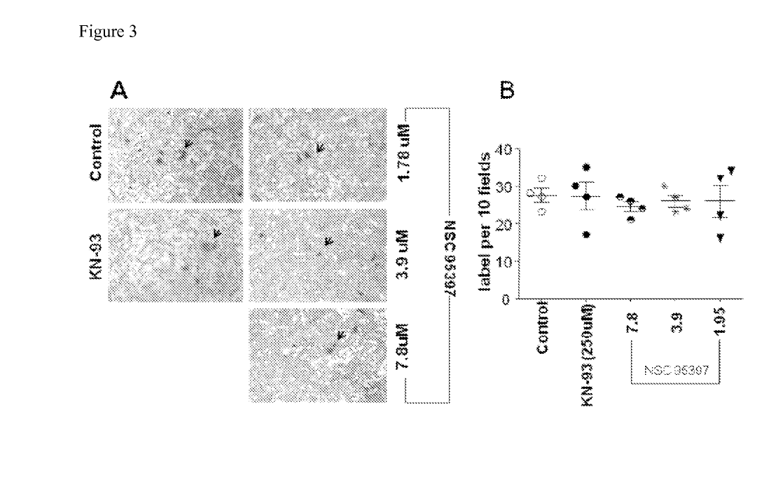

CDC25B and CaMKIIB inhibitors have an additive effect in preventing influenza replication. To determine if KN-93 or NSC 95397 inhibition of influenza virus replication is linked inhibition of virus infection, BEAS2B cells were treated with various drug concentrations and subsequently infected (MOI=0.5) with A/WSN/33, and 24 hpi, the cells were fixed to detect the number of NP-staining, influenza infected cells (FIG. 3). Influenza virus infection of BEAS2B cells treated with KN-93 (250 uM), or various concentrations of NSC 95397, was not perceptibly affected as indicated by similar numbers of NP-staining cells 24 hours pi compared to control treated cells, suggesting that neither drug appreciably affects virus binding or infection.

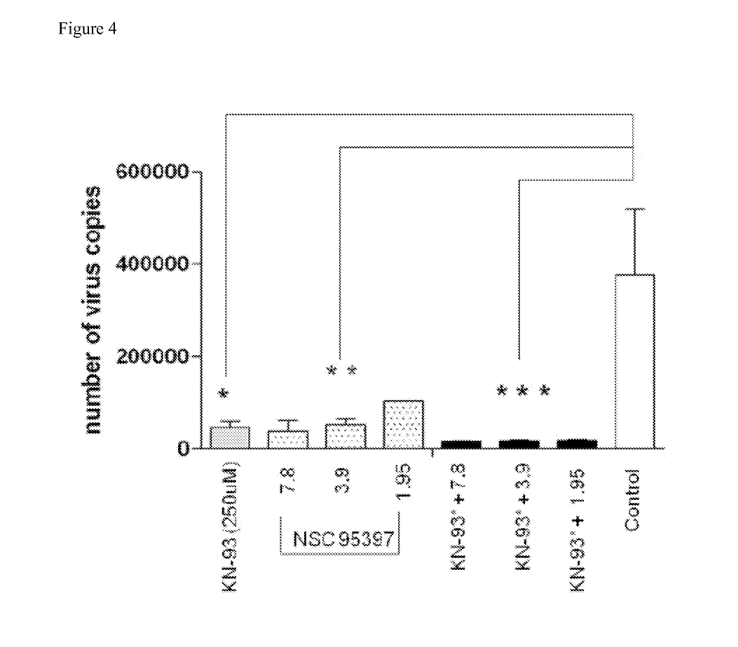

To identify if KN-93 or NSC95397 had additive or synergistic effects in reducing influenza virus replication, BEAS2B cells were prophylactically treated with various concentrations of KN-93 and/or NSC 95397 before infection. Pretreatment of BEAS2B cells with suboptimal concentrations of KN-93 (250 uM; FIG. 1B) and NSC 95397 (1.95 uM; FIG. 2C), respectively had an additive effect on the inhibition of influenza virus replication that was statistically significant (p<0.05) compared to the effect of individual drug treatment alone, and significantly different (p<0.01) than control treatment (FIG. 4). This suggests that each compound likely affects different host cell pathways which are being co-opted by influenza virus for replication, indicating that targeting of CAMKIIB and CDC25B might increase drug efficacy against influenza virus.

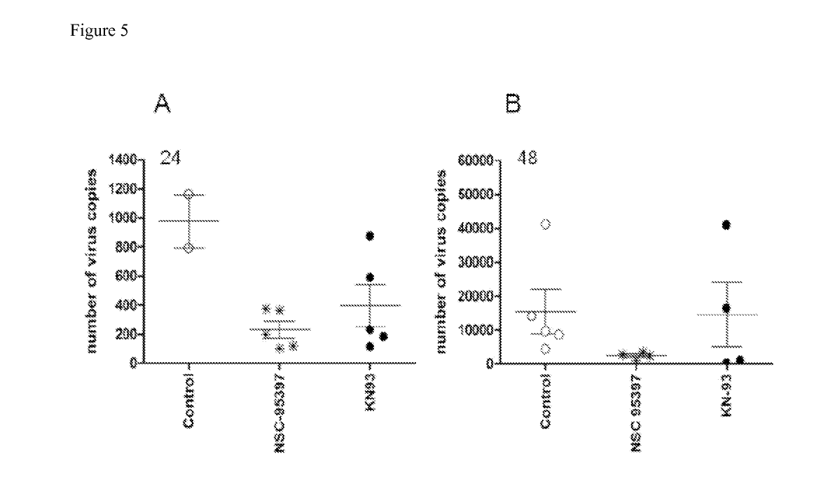

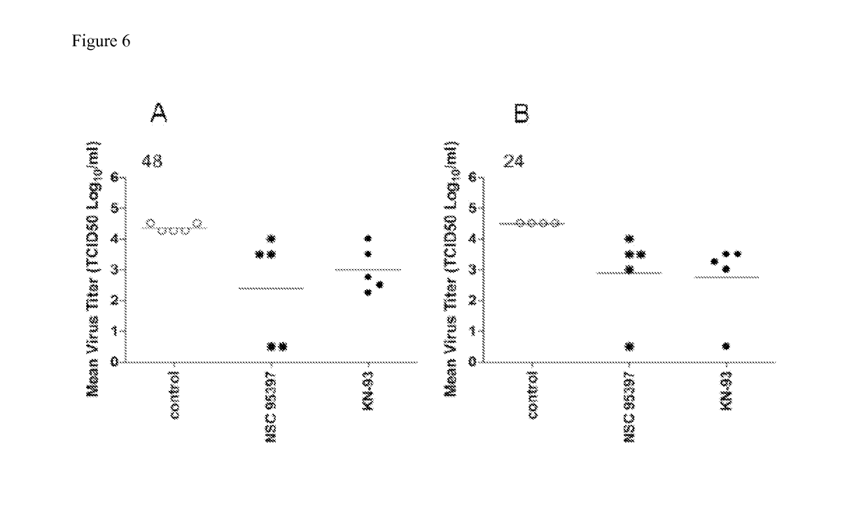

Prophylactic treatment of mice with NSC 95397 or KN-93 inhibits A/WSN/33 and A/CA/04/09 influenza virus replication. To determine if prophylactic treatment with KN-93 or NSC 95397 would shield mice from influenza virus replication, mice were prophylactically treated with NSC 95397 (2.5 mg/kg) or KN-93 (10 mg/kg) for 24 h (FIG. 5A) or 48 h (FIG. 5B) before infection with A/WSN/33 (FIG. 5), or the 2009 pandemic influenza virus, A/CA/04/09 (FIG. 6). Mice treated with drugs only showed no apparent toxicity, no observable adverse events, and had good appetence and appearance. Lungs removed from mice treated with NSC 95397 (2.5 mg/kg) or KN-93 (10 mg/kg) at 24 h and 48 h post-treatment were evaluated and showed down-modulation of CDC25B and CAMKIIB gene expression, respectively by RT-PCR.

Mice prophylactically treated with NSC 95397 (2.5 mg/kg) for 24 h (FIG. 5A) or 48 h (FIG. 5B) were significantly (p<0.05) resistant to A/WSN/33virus replication compared to the control as determined by influenza M gene copy number. Mice prophylactically treated with KN-93 (10 mg/kg) for 24 h were also significantly (p<0.05) shielded from A/WSN/33 replication, but less so at 48 h post-treatment compared to the control (FIG. 5B). These differences likely relate to pharmacokinetic differences. Similarly, mice prophylactically treated with NSC 95397 (2.5 mg/kg) for 24 h (FIG. 6A) or 48 h (FIG. 6B) were significantly (p<0.05) resistant to A/CA/04/09 replication compared to the controls as determined by the levels (TCID₅₀) of infectious virus isolated from the lungs of the mice. Prophylactic treatment with NSC-95367 appeared slightly more effective at 48 h compared to KN-93 (FIG. 6A), while similar effects were observed for both drugs at 24 post-treatments (FIG. 6B). Treatment of A/CA/04/09 infected mice at 24 h or 48 hpi with NSC 95397 (2.5 mg/kg) or KN-93 (10 mg/kg) had no significant (p<0.05) effect in reducing lung virus titers compared to the control.

### Discussion

There are limited influenza drugs available, and few new drug therapies or approaches reported to control influenza virus replication (Min and Subbarao, 2010, Nat Biotechnol 28:239-40). However, as investigators have begun to harness the power of RNAi, a greater understanding of how influenza viruses co-opts host cell pathways to facilitate replication is being uncovered (Konig et al., 2009, Nature 463:813-7, Watanabe et al., 2010, Cell Host Microbe 7:427-39), and this is now opening new avenues for drug targeting and repurposing of drugs that are known to target specific host cell pathways. This present study arises from earlier work by our group that identified and validated mammalian host genes in A549 type II respiratory epithelial cells required for A/WSN/33 replication using a high throughput RNAi siGENOME screening approach similar to that described by others (Brass et al., 2009, Cell 139:1243-54, Hao et al., 2008, Nature 454:890-3, Karlas et al., 2010, Nature 463:818-22, Konig et al., 2009, Nature 463:813-7, Shapira et al., 2009, Cell 139:1255-67, Sui et al., 2009, Virology 387:473-81). One gene in the kinome subfamily, i.e. Ca-Calmodulin kinase 2b (CAMKIIB) (Thiel et al., 1988, Proc Natl Acad Sci USA 85:6337-41), when silenced by RNAi was associated with dramatically reduced A/WSN/33 replication in A549 cells. Interestingly, its specific inhibitor, KN93, was previously shown to prevent virus RNA transcription in culture cells (Konig et al., 2009, Nature 463:813-7). Our laboratory also identified the cell division cycle 25 (CDC25) gene, a member of the CDC25 family of phosphatases, as critical for A/WSN/33 replication in A549 cells.

In this study, we show that inhibition of CAMKIIB using the inhibitor KN-93 prevents A/WSN/33 replication in BEAS2B cells. In addition, we show that the CDC25B inhibitor, NSC 95397, also prevents influenza replication in BEAS2B cells. While neither inhibitor appears to affect virus entry and infection, the CDC25B and CAMKIIB inhibitors clearly inhibit virus replication reducing the number of M gene copies, and when used together, have an additive effect in the inhibition of virus replication. Importantly, prophylactic treatment of mice with micromolar concentrations of NSC 95397 or KN-93 reduced A/WSN/33 and A/CA/04/09 replication in the lungs of mice as indicated by decreased M gene copy numbers, and by decreased levels of infectious virus compared to controls.

The inhibitor, NSC 95397, a p-naphthoquinone, is the most potent CDC25 inhibitor described to date (Han et al., 2004, The Journal of pharmacology and experimental therapeutics 309:64-70, Lazo et al., 2002, Mol Pharmacol 61:720-8). It blocks the G2-to-M transition causing growth arrest of several human carcinoma cell lines. These quinolinediones function by covalently modifying serines on the active site of CDC25 and thereby inhibit its phosphatase activity (Kristjansdottir and Rudolph, 2004, Chem Biol 11:1043-51). A number of novel competitive inhibitors have recently been discovered that specifically act on CDC25 phosphatases blocking cell cycle progression leading to increased phosphorylation of Cdk/cyclin complexes (Kristjansdottir and Rudolph, 2004, Chem Biol 11:1043-51). Based on the efficacious activity of NSC 95397 shown in this study, it is likely that many of these novel competitive inhibitors would also inhibit influenza virus replication.

The mechanism by which CDC25 inhibits influenza virus replication remains unclear. One possibility is that a decrease of CDC25 phosphatase activity could result in over-activation of its target, i.e. the CDK-cyclin complexes affecting influenza virus replication. Many RNA and DNA viruses depend on the host cell cycle for replication, with some like Simian virus 40 (DeCaprio et al., 1988, Cell 54:275-83) and adenovirus (Eckner et al., 1994, Genes & Development 8:869-84) encoding proteins that promote the entrance of cell cycle for support viral synthesis. Similarly, human immunodeficiency virus type 1 (HIV-1) encodes viral proteins that induce cell cycle arrest in G2/M phase (Goh et al., 1998, Nature medicine 4:65-71). Influenza virus infection has also been shown to result in G0/G1-phase accumulation of infected cells caused by the prevention of cell cycle entry from G0/G1 into S phase, and this appears linked to changes levels of p21, cyclin E, and cyclin D1 (He et al., 2010, Journal of virology 84:12832-40). Influenza virus-induced cell cycle arrest causes more efficient viral protein expression and progeny virus production, thus benefiting transcription, translation, and virus assembly efficiencies (He et al., 2010, Journal of virology 84:12832-40). The exact viral protein that is responsible for the observed cell cycle arrest in G0/G1 phase is still unclear. NS1, NA, and PB1 have all been associated with apoptosis (Chen et al., 2001, Nature Medicine, 7:1306-12, Lam et al., 2008, Journal of virology 82:2741-51, Mohsin et al., 2002, Virus research 85:123-31, Schultz-Chemy et al., 2001, Journal of virology 75:7875-81). It is likely that more than one viral protein may be involved in host cell cycle modulation. For example, it may be possible that the transcription activity of the polymerase complex is more active in the G0/G1 phase, or in the S and G2/M phases, and the CDC25 inhibitor may modulate virus replication via interfering with cell cycle. However, it is also possible that cell cycle arrest during influenza virus infection prevents early death of infected cells.

Taken together, the findings from this study show that a better understanding of the host genes required for influenza virus replication can provide critical information about host cell pathways co-opted by influenza virus, and this in turn can be used to repurpose exiting drugs to inhibit gene expression and ultimately virus replication. The studies performed here used BEAS2B cells that are biosimilar to normal bronchial epithelium and corroborated findings in a mouse model. This is important as it shows that at least for CDC25B and CAMKIIB genes that siGENOME screens can be used to identify host genes critical for influenza virus replication and that these findings can be translated to BEAS2B cells as well as murine studies, features that should hasten novel drug anti-viral discovery for influenza virus.

### Example 2

In the process of screening siRNAs to determine the effect of gene silencing on influenza virus (A/WSN/33) replication, it was found that silencing the SLC22A8 gene in human type II respiratory epithelial (A549) cells inhibited influenza virus replication. Pathway analysis suggested this gene could be targeted by an existing drug, and that repurposing this drug may offer an avenue for antiviral therapeutics. Therefore, the SLC22A8 gene was targeted as a potential point of control for protection against influenza. Further research indicated that 4-(dipropylsulfamoyl)benzoic acid, commonly known under the brand name as Probenecid, serves as a chemical inhibitor of SLC22A8 gene. Because this drug is already on the market, having been approved by the FDA for use in the control of gout and for the support of antibiotic therapies, many of the challenges related to getting this drug to the market have already been addressed. This summary details the methods, to date, by which this lab has shown Probenecid to be efficacious against influenza infection.

### Abstract

Influenza A viruses continue to have a major health impact worldwide affecting people of all ages, but have the greatest effect on the elderly, young children, and those vulnerable with underlying conditions. The most effective means for controlling infection is vaccination; however, there is a need for anti-viral drug options. Influenza M2 inhibitor and neuraminidase inhibitor drugs are available but drug resistance has emerged and spread. Research efforts have recently focused on identification of host genes that are essential for influenza replication as they offer potential targets for drug discovery and are refractory to the development of resistance. Using RNA interference and high-throughput genome-wide assays to identify candidate host genes that are required during influenza replication, we identified the SLC22A8 gene, which encodes a protein involved in the sodium-independent transport and excretion of organic anions and is an integral membrane protein as an important host factor required for virus replication. In this study, we show that SLC22A8 gene expression in human bronchoepithelial (A549) cells, and in the lungs of BALB/c mice, can be silenced with Probenecid, which is a uricosuric agent, to inhibit influenza A virus replication without causing toxicity, suggesting targeting of SLC22A8 gene could be a therapeutic approach to treat influenza infection.

### Materials and Methods

Cells and virus stocks. A549 (ATCC CCL-185) type II respiratory epithelial cells were maintained in DMEM medium containing 5% heat inactivated FBS (HyClone, Logan, Utah) at 37° C. in a 5% CO2 incubator. MDCK cells (ATCC CCL-34) was also were maintained the same conditions. A/WSN/33 (H1N1) influenza virus has the ability to replicate without the need for exogenous trypsin (Someya et al., 1990, Biochem Biophys Res Comm., 169:148-152). For the validation studies, A/New Calcdonia/20/99 (H1N1) influenza virus was also used. All viruses were propagated in 9-day-old embryonated chicken eggs as previously described (Woolcock, 2008, Methods Mol. Biol., 436:35-46). Viruses were titrated in MDCK cells and titers calculated by the method developed by Reed and Muench (Reed and Meunch. 1938, Am. J. 488 Hyg 27:493-497).

Human drug target library screen. A primary screen using four pooled siRNAs to target each gene of the 4795 genes in the human drug target library (SMARTpool; Dharmacon ThermoFisher, Lafayette, Colo.) was performed using type II human alveolar pneumocytes (A549 cells) and A/WSN/33 influenza virus similar to a method previously described (Konig et al., 2009, Nature 463: 813-817). In all studies, a siRNA targeting the MEK gene (siMEK) was used to control for the transfection efficiency and host gene silencing. A non-targeting siRNA control (siNEG) was also used in all assays. A549 cells were reverse transfected with siRNA. Lyophilized siRNAs in 96-well plates were diluted with HBSS (HyClone, Logan, Utah) and allowed to incubate for 5 minutes. Dharmafect-1 transfection reagent (Lafayette, Colo.) and HBSS were added such that each well received 0.04 ml of transfection reagent and 0.096 ml of HBSS. The siRNA/transfection reagent mix was allowed to incubate for 20 minutes at room temperature after which 0.08 ml of 1.5×10⁴ A549 cells suspended in DMEM/5% FBS was added to each well and the plate incubated at 37° C. in 5% CO2 for 48 hours before infection with influenza A/WSN/33 (MOI=0.001). The amount of infectious virus was measured 48 hpi by titration of A549 cell supernatant on MDCK cells, and the results normalized to siNEG-treated cells. The infected A549 cells were analyzed for the presence of influenza nucleoprotein (NP) by immunochemistry staining. All assays were run in duplicate and the entire screen assay was repeated three times independently.

NP staining the cells were fixed with cold methanol:acetone (80:20) for 15 min and stained with anti-NP monoclonal antibody (5 ug/ml; H16-L10-4R5) and the antibody staining detected using Alexa Fluor 488 labeled goat-anti mouse IgG (1 ug/ml; Invitrogen, Carlsbad, Calif.). Cells were counterstained with DAPI (2 ug/ml) (Invitrogen, Carlsbad, Calif.) and visualized the cells with virus by immunofluorescent microscopy (20×, EVOS digital inverted fluorescent microscope, Advanced Microscopy Group, Bothell, Wash.) or Cellomics ArrayScan system (Thermo Fisher Scientific), that is an automated fluorescent microscope coupled with image and analytical software that can autonomously record the size, location, and fluorescent intensity (in several channels) of a 96-well plate.

Inhibition assays in vitro. A549 cells were pretreated with decreasing concentrations of probenecid for 24 h before infection. Probenecid (Invitrogen) was dissolved in PBS. The cellular toxicity was determined by the ToxiLight BiAssay kit assay (Lonza, Walkerville, Md., USA). A/WSN/33 of 0.001 or A/New Calcdonia of 0.05 MOI was added to the wells (volume of virus=10% of total volume) without changing medium. At 24 and 48 hpi, supernatant was removed out and remaining cells in triplicates were isolated mRNA for detection of virus replication and gene expression by qRT-PCR or the cells were fixed for NP staining. Qiagen RNAeasy kit (Qiagen, Md., USA) was used for RNA isolation. For detection, the master mix prepared using specific primers for influenza A virus (INFA-F (SS118272-45) 5′ dGACCRATCCTGTCACCTCTGAC (SEQ ID NO:1) and INFA-R(SS118272-46) 5′ dAGGGCATTYTGGACAAAKCGTCTA SEQ ID NO:2) INFA-P (SS118273-01)5′ d FAM TGCAGTCCTCGCTCACTGGGCACG-BHQ-1 (SEQ ID NO:3) (Bioresearch Technologies, Inc., Novato, Calif., provided by CDC). The quantifications were calibrated and optimized using One Step RT-PCR kit (Qiagen) with 30 min at 50° C. for the reverse transcription reaction, 15 min at 95° C. for the Taq inhibitor activation, and PCR amplification with 95° C., 15 sec, 55° C. for 30 sec and a total of 45 cycles in an MX3005P thermocycler (Strategene, Agilent Technologies, Santa Clara, Calif.).

Validation gene expression. The identification of gene was performed by SYBR Green using RT² SYBR Green Master Mix (SABiosciences), SLC22A8 primer (SABiosciences), and GAPDH primer—host keep gene for normalization (BIOsearch Technologies) as the manufacturer protocol. cDNA synthesis was by SuperScript VILO cDNA Synthesis Kit (Invitrogen) for real time PCR. Data was analyzed by calculating 2(−ΔΔCt). Silencing of SLC22A8 gene relative to muck cells were confirmed for each independent experiment.

Animals. BALB/c female mice (6-8 week-old) were obtained from NCI. All experiments and procedures were Institutional Animal Care and Use Committee (IACUC) approved by the IACUC of UGA. The animals were kept alone in cages. They were allowed free access to water and food both before and during the experiments. All experiments were performed with 5 mice per group and repeated independently at least twice.

Drug treatment and infection. Probenecid was dissolved in PBS and injected intraperitoneally at final doses of 10 and 200 mg/kg body weight (about 0.2 ml per animal). Similar studies were done using the same doses but delivering by gavage. As intraperitoneal administration was equally effective and less invasive, the majority of studies were performed in this fashion. The treatments were 24 h or 48 h before infection. Prior to infection, mice were anesthetized with Avertin and infected by intranasal instillation with 50 μl of PBS containing 100 PFU of A/WSN/33 or A/New Calcdonia/20/90 influenza virus. The lungs of the mice were collected at 48 hip for qRT-PCR and TCID50. In some studies, Probenecid was administered at 48 h prior to influenza virus infection by tail vein injection (200 ul) where the following groups were evaluated (5 mice/group): Group 1—Probenecid 5.0 mg/kg; Group 2—Probenecid 0.5 mg/kg; Group 3—Probenecid 0.05 mg/kg; Group 4—Positive Control—KN93 (10 mg/kg); Group 5—Negative Control (PBS); Group 6—Probenecid 5.0 mg/kg no infection (gene silencing only).

Detection of virus replication. qRT-PCR and TCID50 of the lungs. The lungs of infected animals were longitudinally sectioned. One half of the lung was into TRizol (Invitrogen, Carlsbad, Calif., USA) for isolating total RNA. Another half lung was into MEM/without serum media for TCIT50. The qRT-PCR performance for both of virus replication and the gene expression was like in vitro.

Statistical analyses. All statistical analyses was using student's t test by Graphpad Prism software. Data were expressed as means±S.E. Values of P<0.05 were considered significant.

### Results

The development of new antiviral therapeutics requires a greater understanding of the global host response when challenged by different types of viruses. Such knowledge may lead to the identification of novel human genome targets that are shared across multiple viral infections as well as opportunities for re-positioning existing drugs for the treatment of infectious disease.

There has been an increasing interest in drug repositioning in recent years, which is motivated by multiple factors. The average cost of introducing one new drug to the market in developed countries, including the cost of failures, has been estimated to be USD 1.24 billion. In addition, the time required to develop a new drug de novo varies between 10 to 17 years due to regulatory requirements regarding safety, efficacy, and quality, in both, animal studies and clinical trials. Moreover, high attrition rates are also a major concern for pharmaceutical companies. Repositioned drugs have the advantage of decreased development costs and decreased time to market than traditional discovery efforts, due to availability of previously collected pharmacokinetic, toxicology, and safety data. The recent regulatory environment has also become more restrictive, leading to newer and more stringent regulations that a new drug must meet in order to enter the market. Stricter regulations have also led to significant increase in the time and cost of new drug development. It has been estimated that the time required for development of a repositioned drug varies between 3 and 12 years with substantially lower costs, thereby ensuring the repositioning company significant savings in terms of time and capital. For repositioned drugs, as the clinical safety data, pharmacokinetics, and viable dose range are available at the start of a development project, the risks associated with clinical development are significantly reduced with fewer failures in the later stages.

This study used a systematic process involving siGENOME RNAi libraries that target every gene in the human genome to identify genes in host cell pathways critical to influenza virus replication. These measures included candidate dataset filtering followed by QC, differential gene expression and pathway enrichment analyses. The findings lead us to the SLC22A8 gene as being critical for influenza A virus replication in human respiratory epithelial (A549) cells. Our analysis suggests several potential repurposing opportunities for launched drugs against the SLC22A8 gene. This assumption was based on the occurrence of gene in infection models for A/WSN/33, and involvement in a number of relevant pathways related to host response, and those pathways that may encode for known drug targets. Further research indicated that 4-(dipropylsulfamoyl)benzoic acid, commonly known under the brand name as Probenecid, serves as a chemical inhibitor of SLC22A8 gene. Because this drug is already on the market, having been approved by the FDA for use in the control of gout and for the support of antibiotic therapies, many of the challenges related to getting this drug to the market have already been addressed. This summary details the methods, to date, by which this lab has shown Probenecid to be efficacious against influenza infection.

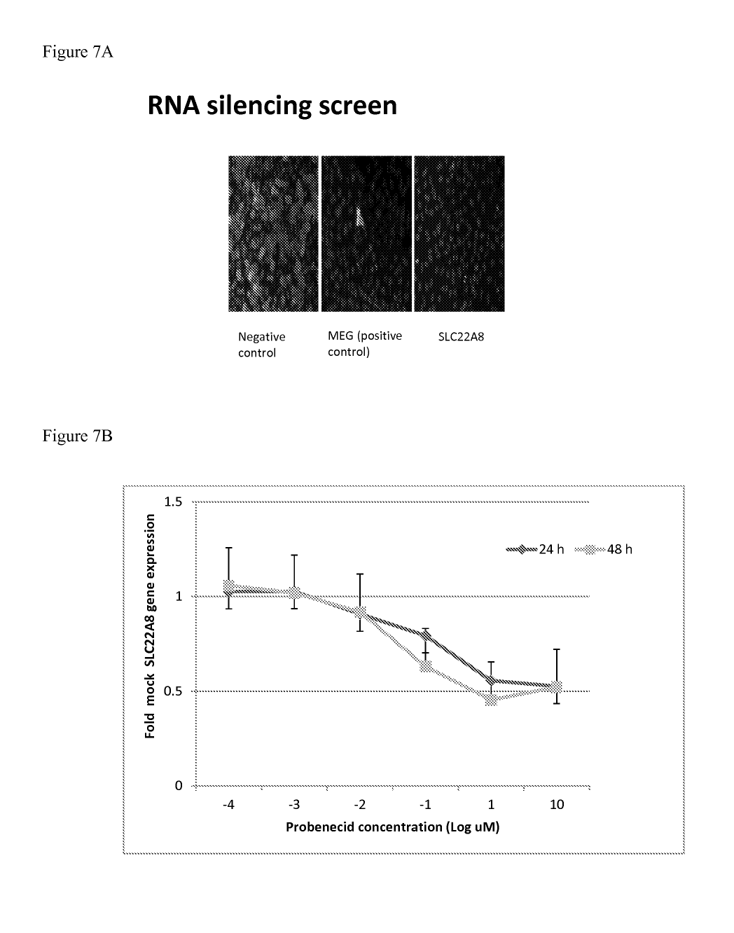

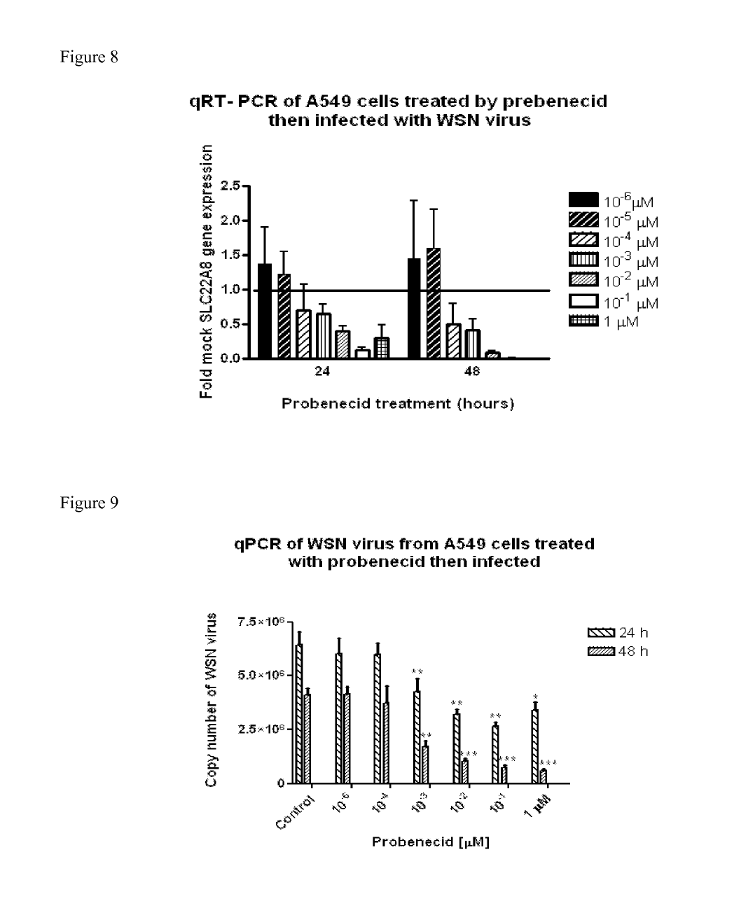

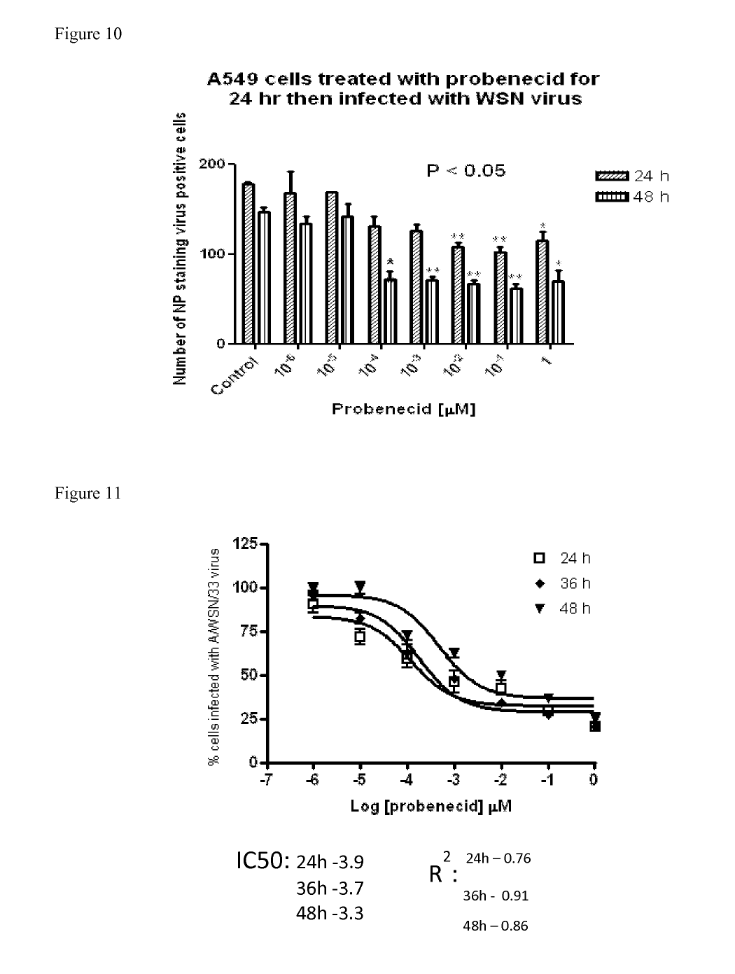

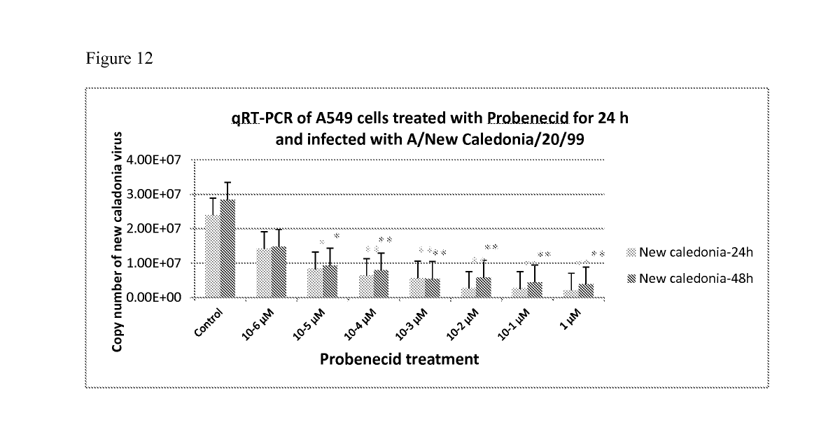

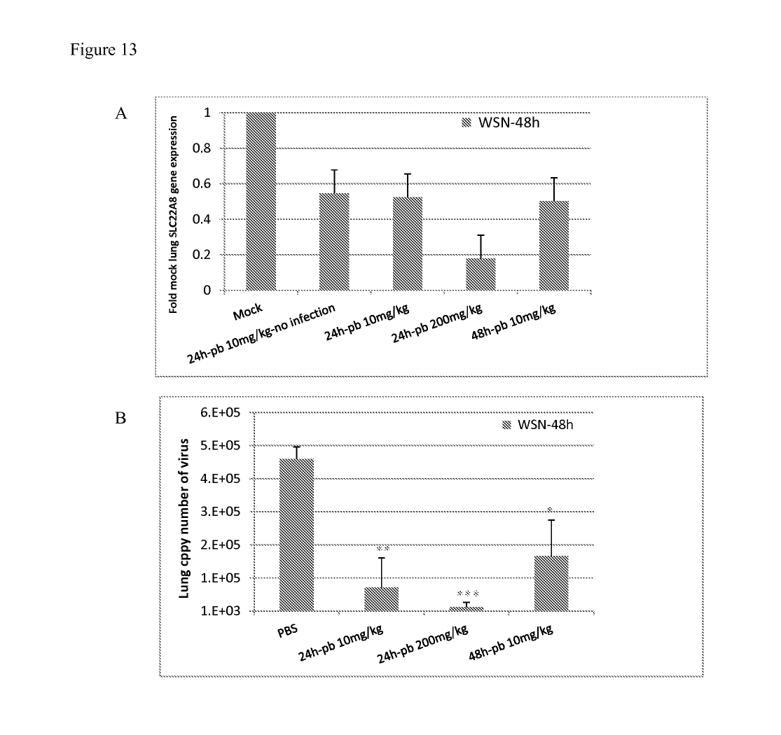

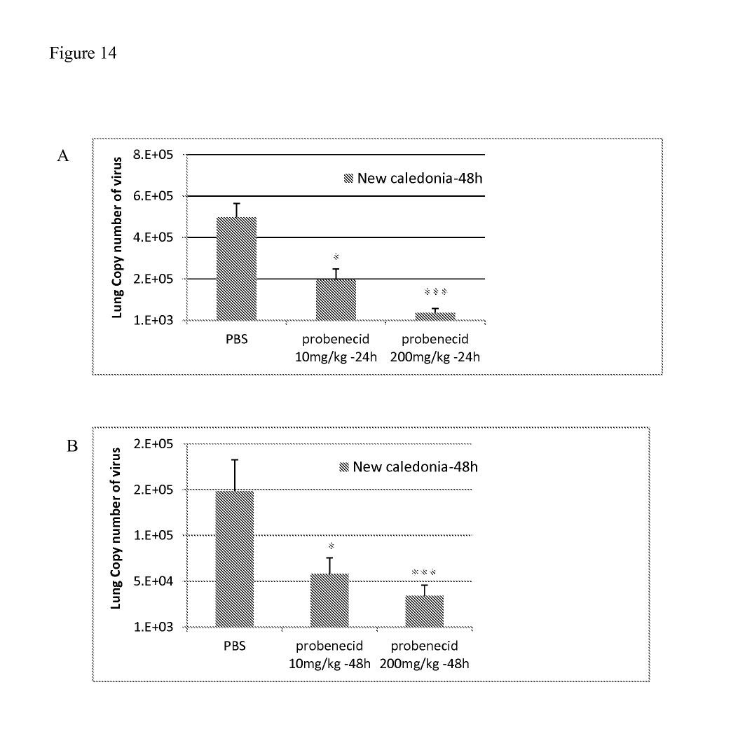

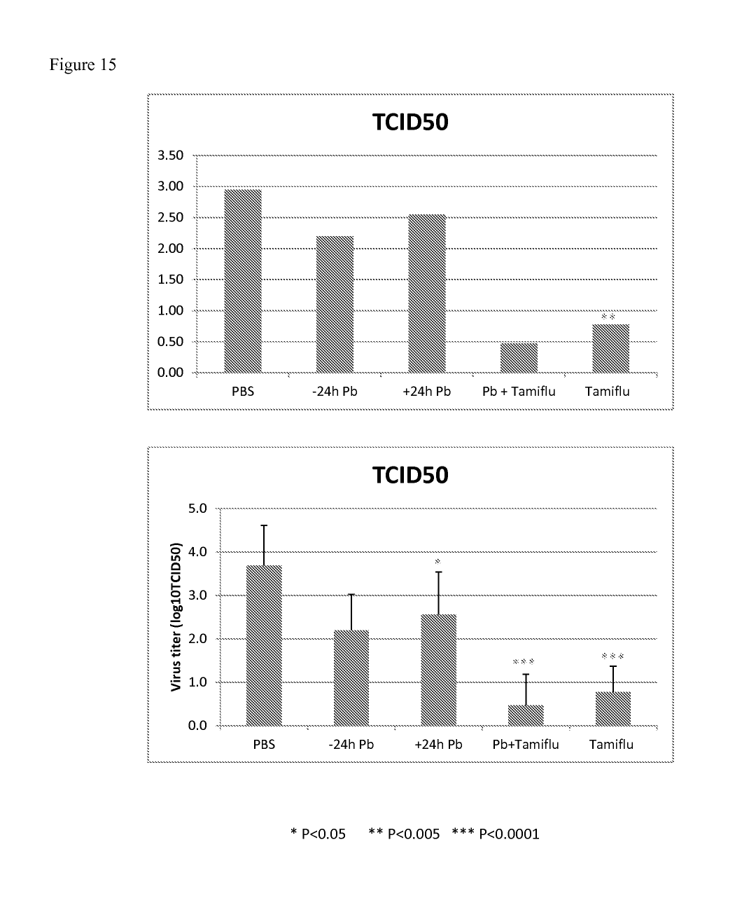

These results in this report show that pharmacologically administered levels of Probenecid effectively reduce SLC22A8 gene expression in A549 cells (FIGS. 7A, 7B, and 8), and that A549 cells treated prophylactically or therapeutically with varying concentrations of Probenecid are refractory to influenza virus replication (FIGS. 9, 10, 11, and 12). We show this level of efficacy for two strains of influenza A virus, i.e. A/WSN/33 and A/New Calcdonia/20/90. Importantly, we show that pharmacologically administered levels of Probenecid effectively reduce SLC22A8 gene expression in 6-8 week old female BALB/c mice, and that mice treated prophylactically or therapeutically with varying concentrations of Probenecid are refractory to influenza virus replication (FIGS. 13 and 14). Further, we show that Probenecid treatment is as effective as Tamiflu treatment in mice and combining treatment of Probenecid and Tamiflu synergized efficacy in preventing influenza virus replication (FIG. 15). This is not unexpected as during World War II, Probenecid was used to extend limited supplies of penicillin, is still used to increase antibiotic concentrations in serious infections, and in one study, Probenecid was shown to more than double blood concentrations of Tamiflu (oseltamivir) (used to combat influenza virus infection (2002, Drug Metab. Dispos., 30(1):13-9).

### Example 3

### Targeting Cell Division Cycle 25 Homolog B (CDC25B) to Regulate Influenza Virus Replication

Influenza virus is a worldwide global health concern causing seasonal morbidity, mortality, and economic burden. Chemotherapeutics is available however rapid emergence of drug resistant influenza strains has reduced their efficacy, thus there is a need to discover novel anti-viral agents. In this study, RNA interference (RNAi) was used to screen host genes required for influenza virus replication. One pro-influenza virus host gene identified was dual-specificity phosphatase cell division cycle 25 B (CDC25B). RNAi of CDC25B resulted in reduced influenza A virus replication, and a CDC25B small molecule inhibitor (NSC95397) inhibited influenza A virus replication in dose-dependent fashion. Viral RNA synthesis was reduced by NSC95397 in favor of increased interferon beta (IFNβ) expression, and NSC95397 was found to interfere with nuclear localization and chromatin association of NS1, and influenza virus protein. As NS1 has been shown to be chromatin-associated to suppress host transcription, it is likely that CDC25B supports NS1 nuclear function to hijack host transcription machinery in favor of viral RNA synthesis, a process that is blocked by NSC95397. Importantly, NSC95397 treatment protects mice against lethal influenza virus challenge. The findings establish CDC25B as a pro-influenza A host factor that may be targeted as a novel influenza A therapeutic strategy.

### Materials and Methods

Cells and viruses. Human type-II respiratory epithelial (A549) cells (ATCC, CCL-185) and Madin-Darby Canine Kidney (MDCK) cells (ATCC, CCL-34) were cultured in Dulbecco's modified Eagle's medium (DMEM), supplemented with 5% heat-inactivated FBS (HyClone, Utah) in a 37° C. incubator with 5% CO₂. Human bronchoepithelial (BEAS2B) cells (ATCC CRL-9609) were maintained in Bronchial Epithelial Basal medium (BEBM; Lonza, Md.) supplemented with 30 μg/ml bovine pituitary extract, 0.5 μg/ml hydrocortisone, 0.5 ng/ml human recombinant epidermal growth factor, 0.5 μg/ml epinephrine, 10 μg/ml transferrin, 5 μg/ml insulin, 0.1 ng/ml retinoic acid, and 6.5 ng/ml triiodothyronine (BEGM SingleQuots; Lonza, Md.) at 37° C. in a 5% CO2 incubator. Influenza A virus, A/WSN/33(H1N1), was propagated in 9 day-old embryonated chicken eggs obtained from a specific-pathogen-free leghorn chicken flock (Sunrise Farms, N.Y.). The allantoic fluid from this parental stock was tested for hemagglutinating (HA) activity. HA positive allantoic fluids were pooled, aliquoted, and stored at −80° C. until use. A mutant influenza A/WSN/33 virus expressing defective RNA-binding NS1 (NS1 R38AK41A) was a kind gift from Dr. Adolfo García-Sastre (Mount Sinai School of Medicine, N.Y.) and was passaged once in MDCK cells. Mutations were confirmed by sequence analysis. MDCK cells were used to determine the titer of the A/WSN/33 stock virus and from culture supernatant of infected cells as previously described (Gaush et al., 1968. Applied microbiology 16:588-594, Szretter et al., 2005. Influenza: Propagation, Quantification, and Storage, Current Protocols in Microbiology. John Wiley & Sons, Inc.).

RNAi transfection. A primary RNAi screen was performed using four pooled siRNAs to target each gene of the 4,795 genes in the human drug target library (SMARTpool; Dharmacon ThermoFisher, CO) using A549 cells infected with influenza A/WSN/33 virus as previously described (Konig et al., 2010. Nature 463:813-817, Meliopoulos et al., 2012. PloS one 7:e37169). For CDC25B validation study, individual siRNAs targeting human CDC25B and a non-targeting siRNA were used (Dharmacon ThermoFisher, CO). A549 cells were reverse transfected with siRNA using DharmaFECT-1 reagent (Dharmacon, Colo.) as previously described (Meliopoulos et al., 2012. PloS one 7:e37169). Transfections were carried out for 48 hours to allow maximal expression knock-down before cells were infected with influenza A/WSN/33 at a MOI=0.001. The level of infectious virus was measured 48 hours post-infection (hpi) by titration of A549 cells supernatants on MDCK cells (Reed et al., 1938. American Journal of Epidemiology 27:493-497). In addition, A549 cells monolayer on culture plates were fixed and analyzed for the presence of influenza NP by immunofluorescence staining as described below. Transfected cells were also collected to assess CDC25B gene expression knock down using the qRT-PCR method described below.

In vitro inhibition assays. NSC95697 (2,3-bis-[2-hydroxyethylsulfanyl]-[1,4]naphthoquinone) (TOCRIS Bioscience, MI) was dissolved in DMSO and serially diluted in BEBM media. For dose-response virus inhibition experiments, cells were washed with phosphate-buffered saline (PBS) once prior to titration of NSC95607 using the Hewlett-Packard (HP) D300 Digital Dispenser (Tecan, N.C.) (Jones et al., 2013. Journal of laboratory automation) 1 hour before infection. For time-of-addition experiments, 2 μM of NSC95397 were added at different time points pre- or post-infection. Where indicated, cells were subsequently infected with influenza A/WSN/33 at MOI=0.1 or 1. At the indicated time points, cells were fixed with 4% formaldehyde for subsequent immunostaining, collected for total RNA isolation using Qiagen RNAeasy kit (Qiagen, Md.) for gene expression analyses, or collected for protein analyses using immunoblotting. Furthermore, culture supernatant was collected for IAV titration in MDCK cells and cytotoxic analysis. Cellular toxicity was determined by measuring adenylate kinase release using the ToxiLight Bioassay kit (Lonza, Md.).

Gene expression analyses. For measurement of influenza A viral copy number, total RNA collected from infected A549, BEAS2B cells, or lungs of infected mice were used for quantitative realtime-PCR (qRT-PCR) assay using the OneStep RT-PCR kit (Qiagen, Md.).

Universal influenza primers-probe set was used for amplification and detection of influenza A virus RNA (InfA forward (SS118272-45), InfA reverse (SS118272-46), and InfA probe (SS118273-01); Bioresearch Technologies, Inc., CA) as previously described (Perwitasari et al., 2012. Antimicrobial agents and chemotherapy).

For strand-specific IAV qRT-PCR analyses, primers specific for IAV segment 5 cRNA, mRNA, and vRNA containing additional 18-20 nucleotide tag unrelated to IAV at the 5′ end were used for increased specificity to distinguish the three different IAV RNA species as described previously (Kawakami et al., 2011. Journal of Virological Methods 173:1-6). Briefly, equal amounts of total RNA from infected cells were used to synthesize cDNA complementary to the three types of IAV RNA using Verso cDNA Synthesis Kit (Thermo Scientific, MA). Quantitative PCR analysis was performed using RT² SYBR Green qPCR Master Mix (SABioscience, MD) and primer sets specific to the corresponding IAV RNA species in MX3005P thermocycler. To assess CDC25B and IFNβ gene expressions, cDNA were synthesized using random hexamers as primer (Thermo Scientific, MA). cDNA were subsequently used for quantitative PCR amplifications using CDC25B, IFNβ, and GAPDH gene specific primers and RT² SYBR Green qPCR Master Mix (SABioscience, MD) in MX3005P thermocycler as previously described (Perwitasari et al., 2012. Antimicrobial agents and chemotherapy). Abundance of viral RNA, CDC25B, and IFNβ gene expressions were normalized to GAPDH and their expressions relative to mock-treated samples were calculated using 2^{(-ΔΔCt)} formula.

Immunofluorescence staining. Cells were fixed with 4% formaldehyde for 10 minutes, blocked in 3% BSA, and incubated with primary antibodies: mouse anti-NP and rabbit anti-NS1 (Pierce antibodies, Thermo Scientific, MA), followed by incubation with appropriate secondary antibodies: Alexa 488-conjugated goat anti-mouse and Alexa 546-conjugated goat anti-rabbit (1 μg/ml; Invitrogen, CA), and DAPI counterstain (2 μg/ml; Invitrogen, CA). Cells were visualized using EVOS fluorescent imaging system (Advanced Microscopy Group, WA). For high content imaging, cells were visualized and counted using Cellomics ArrayScan system (Thermo Scientific, MA) with proprietary image and analytical software.

Protein isolation, subcellular fractionation, and immunoblot analysis. To evaluate total protein expressions following NSC95397 treatment, cells were lysed in radioimmune precipitation assay (RIPA) buffer [50 mM Tris HCl (pH 7.5), 150 mM NaCl, 0.5% sodium deoxycholate, 1% Nonidet P-40, 1 mM EDTA, and 0.1% sodium dodecyl sulfate (SDS)] supplemented with Halt protease and phosphatase inhibitor cocktail (Thermo Scientific, MA), followed by 4° C. centrifugation at 16,000×g for 10 minutes to clarify lysate. For protein fractionation experiments, cell pellet were subjected to subcellular fractionation (Pierce, Thermo Scientific, MA) to biochemically isolate cytoplasmic, soluble nuclear, and chromatin-bound proteins. Briefly, for isolation of chromatin proteins non-soluble nuclear pellets were digested with micrococcal nuclease, supplied in manufacturer's kit, to digest chromatin and extract chromatin-bound proteins.

Equivalent protein amounts were diluted in SDS sample buffer [for 4× buffer: 40% glycerol, 240 mM Tris/HCl (pH 6.8), 8% SDS, 0.04% bromophenol blue, 5% β-mercaptoethanol], boiled, and resolved by SDS-polyacrylamide gel electrophoresis followed by immunoblotting. Primary antibodies used for immunoblot analyses were: mouse anti-NP, rabbit anti-NS1 (Pierce antibodies, Thermo Scientific, MA), mouse anti-NS1 (Santa Cruz Biotechnology, TX), mouse anti-CDK1 and rabbit anti-CDK1 pY15 (Abcam, Mass.), rabbit anti-ERK1/2, ERK 1/2 pT202/Y204 and histone H3 (Cell Signaling Technology, MA), and rabbit anti-GAPDH (Millipore, Mass.). Horseradish peroxidase-conjugated goat anti-rabbit and anti-mouse antibodies (Sigma-Aldrich, MO) were used as secondary antibodies. Protein bands were visualized following addition of SuperSignal West Dura Extended Duration Substrate (Pierce, Thermo Scientific, MA) and visualized using the Fluor Chem-E western imaging system (ProteinSimple, CA).

Mice and infections. BALB/c female mice (8-10 week-old) were obtained from NCI. All experiments and procedures were approved by the Institutional Animal Care and Use Committee (IACUC) of the University of Georgia. Mice were treated with DMSO or 2.5 mg/kg NSC95397 orally by gavage at 24-hours pre- or post-infection.

Prior to virus inoculation, mice were anesthetized with Avertin and intranasally infected with lethal (10³ PFU) or sub-lethal (70 PFU) of influenza A/WSN/33 in 50 μl PBS. Body weight and survival were evaluated daily for 14-days. For assessment of lung viral burden, lungs of infected mice were collected at 72 hpi and homogenized in TRizol (Invitrogen, CA) for total RNA isolation. Five nanograms of total RNA were used for assessment of IAV copy number by qRT-PCR analysis as described above. Experiments were performed with ten mice per group.

Statistical analyses. The Student's t-test was used throughout, except for the survival curves where the One-Way ANOVA and the Mantel-Cox test were employed. p-values<0.05 are considered significant.

### Results

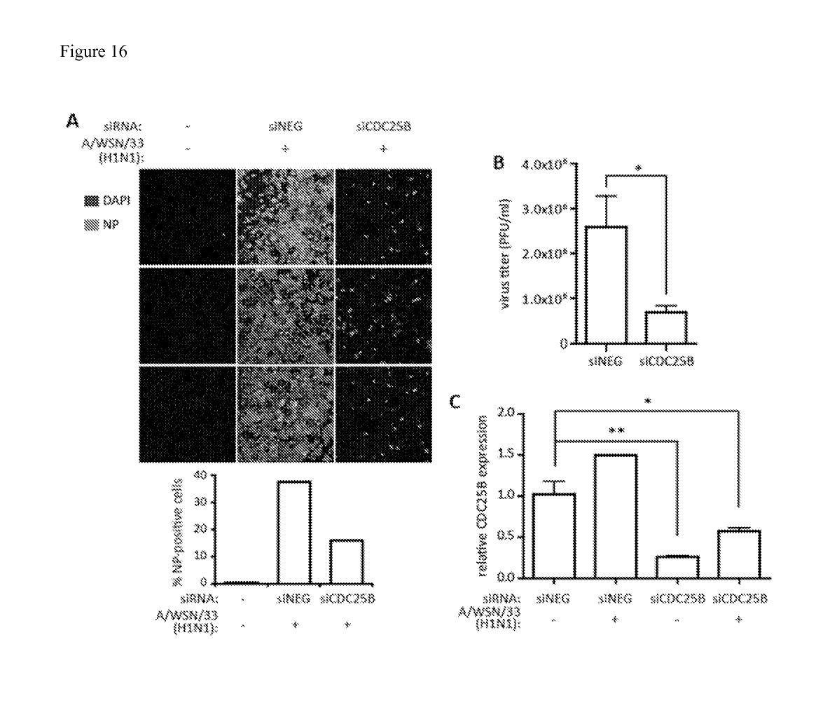

CDC25B is a pro-influenza virus host factor. RNAi screening that was performed identified numerous host cell genes that are required for IAV replication (Perwitasari et al., 2012. Antimicrobial agents and chemotherapy, Meliopoulos et al., 2012. FASEB J., 26:1372-1386, Meliopoulos et al., 2012. PloS one 7:e37169). One of the genes identified was CDC25B, a member of the dual-specificity CDC25 phosphatases that has been shown to dephosphorylate CDK1 and ERK1/2 (Kristjansdottir et al., 2004. Chem Biol 11:1043-1051, Nemoto et al., 2004. The Prostate 58:95-102). To validate this finding, individual non-pooled siRNA targeting CDC25B (siCDC25B) were used for transfection of A549 cells 48-hours prior to infection with influenza A/WSN/33 (MOI=0.05). siCDC25B transfection resulted in a reduction of influenza A NP-positive cells at 48 hpi compared to non-targeting siRNA (siNEG) transfection as visualized and quantified by high-content imaging (FIG. 16A, quantified in bottom panel). Similarly, siCDC25B-transfected cells also had significant (p<0.05) reduction of influenza A/WSN/33 virus titer in culture supernatant collected at 48 hpi compared to siNEG transfected cells (FIG. 16B). To verify knock-down of CDC25B gene expression following siRNA transfection, mock or A/WSN/33-infected, siRNA-transfected cells were collected for total RNA isolation and qRT-PCR analysis. A549 cells transfected with siCDC25B showed 80% or 45% reduction of CDC25B relative to GAPDH mRNA level for mock (p<0.01) or A/WSN/33-infected cells (p<0.05), respectively, compared to siNEG-transfected, mock-infected cells (FIG. 16C). Interestingly, influenza A/WSN/33 infection resulted in upregulation of CDC25B gene expression, further indicating a role for virus replication.

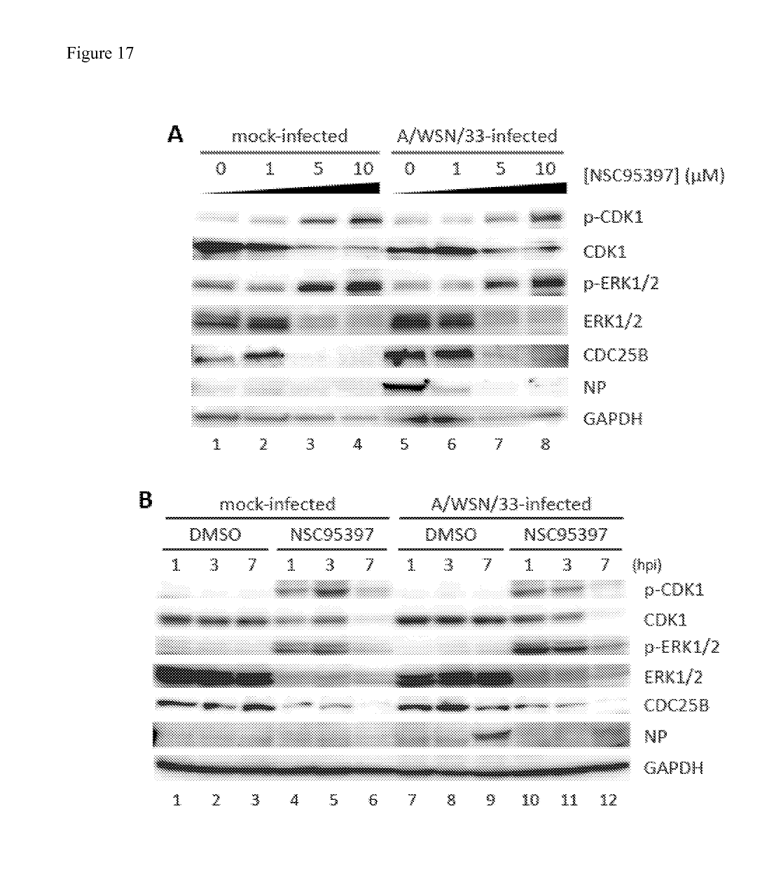

NSC95397 modulates CDC25B phosphatase activity during IAV infection. To further determine if CDC25B is a viable target to limit IAV replication, a selective and irreversible inhibitor of CDC25B phosphatase, i.e. NSC95397 was evaluated (Lazo et al., 2002. Mol Pharmacol 61:720-728, Park et al., 2007. Bioorg Med Chem Lett 17:2351-2354, Peyregne et al., 2005. Molecular Cancer Therapeutics 4:595-602). CDC25B has been found to be overexpressed in carcinoma cells (Bugler et al., 2010. Molecular Cancer 9:29, Boutros et al., 2007. Nature reviews. Cancer 7:495-507); thus non-neoplastic human bronchoepithelial BEAS2B cells were employed in the subsequent studies. To demonstrate that NSC95397 inhibits CDC25B activity in the context of virus infection, levels of phosphorylated CDK1 and ERK1/2, both are known targets of CDC25B phosphatase activity, were evaluated in mock or influenza A/WSN/33-infected BEAS2B cells in presence or absence of NSC95397. BEAS2B cells were pre-treated with DMSO or increasing dose of NSC95397 for one hour prior to mock- or IAV-infection at an MOI=1, and protein lysates were collected at 8 hpi for immunoblot analysis (FIG. 17A). Mock or IAV-infected cells pre-treated with NSC95397 displayed higher levels of phosphorylated CDK1 and ERK1/2 proteins in dose-dependent fashion. Levels of total CDK1 and ERK1/2, as detected by immunoblot assay, appeared to be diminished as levels of phosphorylated CDK1 and ERK1/2 increased. This is presumably due to reduced affinity of CDK1 and ERK1/2 antibodies for phosphorylated CDK1 and ERK1/2, respectively. Consistent with FIG. 16C, CDC25B protein level was upregulated in IAV-infected cells (FIG. 17A, lane 5) compared to mock-infected cells (lane 1) in absence of treatment. However, CDC25B protein level was reduced at high concentrations of NSC95397 regardless of infection (lanes 3-4; 7-8). Importantly, NSC95397-treatment was able to reduce level of influenza NP protein in dose-dependent manner (lanes 5-8), with 71.8% and 81.5% reduction of NP expression for 1 μM and 5 μM NSC95397, respectively.

To further evaluate the kinetic of NSC95397 activity toward CDC25B during IAV infection, BEAS2B cells were pre-treated with DMSO-treated or 5 μM NSC95397 one hour prior to mock- or influenza A/WSN/33 infection at MOI=1 (FIG. 17B). Protein lysates were collected for immunoblotting at 1, 3, or 7 hpi. Phosphorylated CDK1 and ERK1/2 can be readily observed at 2 hours following NSC95397 treatment (1 hpi) in both mock- or IAV-infected cells, and diminished by 8 hours post-treatment (7 hpi) (FIG. 17B, lanes 4-6; 10-12), but not in DMSO-treated cells (lanes 1-3; 7-9). Cells treated with NSC95397 maintained CDC25B protein level to 4 hours post-treatment but diminished by 8 hours (lanes 4-6; 10-12). It is important to note that the increase in CDK1 and ERK1/2 phosphorylation preceded the downregulation of CDC25B protein level, suggesting that NSC95397 acted to inhibit activity of CDC25B to dephosphorylate CDK1 and ERK1/2. However, this modulation of CDC25B by NSC95397 would ultimately result in reduction of CDC25B protein abundance, presumably by affecting its stability. Importantly, while NP protein can be detected at 8 hpi in DMSO-treated cells (lane 9), NSC95397 treatment resulted in diminished NP expression (lane 12), in agreement with the finding in FIG. 17A. These findings demonstrate that inhibition of CDC25B activity by NSC95397 can ultimately lead to reduction of IAV infection.

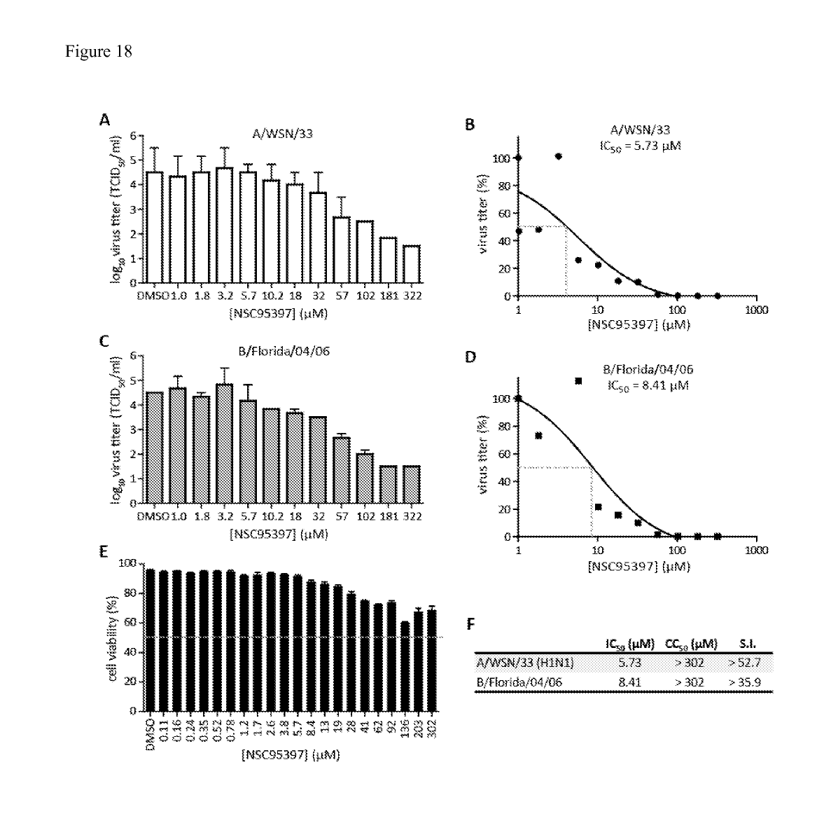

NSC95397 limits IAV replication in BEAS2B cells. To further determine if CDC25B is a druggable target for inhibiting IAV replication, and to further evaluate the mechanism of CDC25B as a pro-influenza gene, efficacy of NSC95397 against influenza A and B viruses was evaluated (FIG. 3). BEAS2B cells pre-treated for 1 hour with NSC95397 and subsequently infected with A/WSN/33 (MOI=0.05) displayed reduced virus titer in culture supernatant at 24 hpi (FIG. 18A). NSC95397 was able to limit influenza A/WSN/33 virus replication in BEAS2B cells in a dose-dependent manner, with 50% inhibitory concentration (IC₅₀) of 5.73 μM (FIG. 18B). Similarly, NSC95397 treatment was able to reduce influenza B/Florida/04/06 titer in dose-dependent manner, with IC₅₀ of 8.41 μM (FIG. 18C-D). NSC95397-treated BEAS2B cells displayed minimal cytotoxicity up to 302 μM, where 50% cellular cytotoxicity (CC₅₀) was not yet observed (CC₅₀>302 μM; FIG. 18E). These results demonstrated efficacy of NSC95397 against representative strain of both influenza A and B viruses, with selectivity indices (S.I.) of >52.7 and >35.9, respectively (FIG. 18F). Taken together, these results further confirmed that CDC25B has a pro-viral role during IAV replication, and that its inhibition by RNAi or by small molecule inhibitor limits IAV infection in vitro.

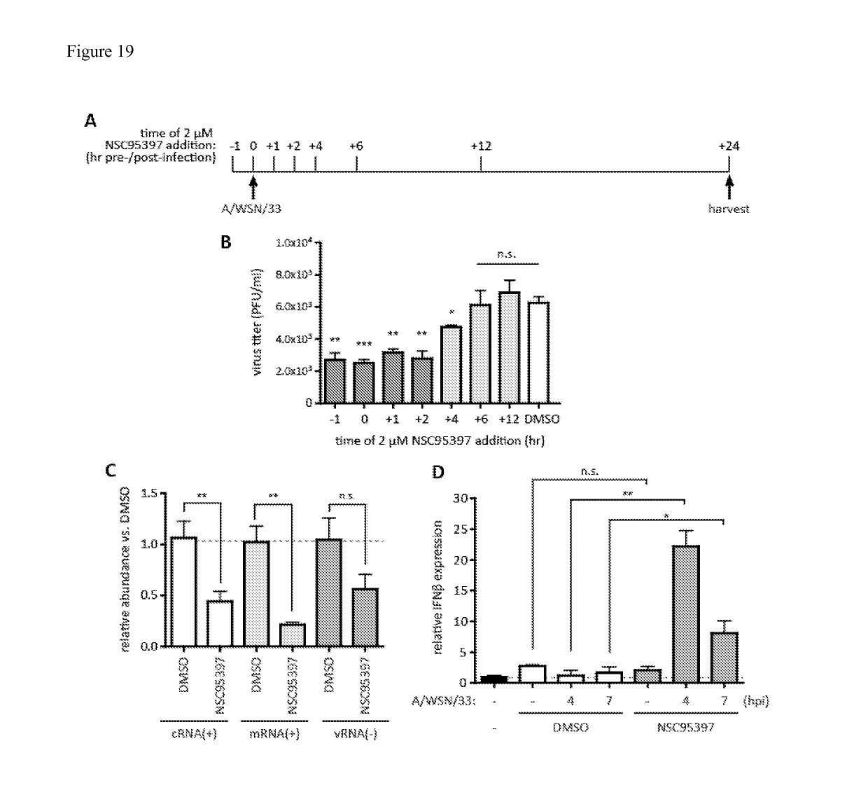

NSC95397 inhibits IAV RNA synthesis and promotes type-I IFN expression. To identify a mechanism of action for NSC95397 inhibition of IAV replication, the point in the virus life cycle inhibited by NSC95397 was determined. To address this, BEAS2B cells were treated with 2 μM NSC95397 at different time points pre- or post-A/WSN/33 infection at MOI=1 (FIG. 19A). Culture supernatant of infected cells was collected at 24 hpi for virus titration in MDCK cells (FIG. 19B). Different periods of 2 μM NSC95397 treatment over 24 hpi had no effect on host cell viability as assessed by ToxiLight bioassay and by phase contrast microscopy. However, a significant reduction in virus titer was evident in cells treated with NSC95397 before 6 hpi indicating that NSC95397 inhibits IAV replication mid-cycle, i.e. when virus RNA replication is occurring in the nucleus. To determine if viral RNA synthesis was inhibited by NSC95397, strand-specific qRT-PCR were employed to evaluate the abundance of viral cRNA, mRNA, and vRNA at 7 hpi (FIG. 19C). Abundance of (+) sense viral cRNA and mRNA were reduced 55% and 80%, respectively, by NSC95397 compared to DMSO-treated cells (p<0.01). Although slight reduction of (−) sense vRNA was also observed in presence of NSC95397, this difference was not statistically significant. Importantly, while abundance of viral RNA was reduced, higher IFNβ expression was observed following NSC95397 treatment compared to DMSO-treated cells (FIG. 19D). A/WSN/33-infected cells treated with DMSO did not show significant increase of IFNβ expression at 4 and 7 hpi, consistent with previous findings on antagonism of host IFN response by IAV. Remarkably, cells infected with A/WSN/33 in presence of 2 μM NSC95397 displayed 23-fold and 10-fold increases of IFNβ mRNA expression at 4 and 7 hpi, respectively. This was a significant increase when compared to DMSO-treated cells at the respective time points post-infection (p<0.01 and p<0.05 for 4 and 7 hpi, respectively). In absence of infection, NSC95397 did not upregulate IFNβ expression when compared to DMSO-treated cells. Furthermore, higher expression of type-III IFN (IFNλ1) was also detected in presence of NSC95397 at 4 hpi (data not shown). Together, these results suggest that NSC95397 inhibits IAV (+) sense RNA synthesis while inducing higher level of host type-I and type-III IFN genes expression.

NSC95397 anti-influenza action is mediated by modulation of viral NS1 function in the nucleus. Since influenza virus is known to antagonize host IFNs responses by inhibiting the RIG-1-like receptor (RLR) signaling pathway through the action of viral NS1 protein, the signaling event downstream of RLR activation, i.e. phosphorylation of interferon regulatory factor (IRF)-3 during the course of IAV infection was evaluated in absence or presence of NSC95397 treatment. BEAS2B cells were pre-treated with DMSO or 5 mM NSC95397 1 hour prior to mock or influenza A/WSN/33 infection (MOI=1). Cells were harvested for protein isolation at 4- and 7-hpi and levels of phosphorylated and total IRF-3 were evaluated by immunoblotting (FIG. 5A). Phosphorylated IRF-3 was detected at 4 hpi and was sustained at 8 hpi in both DMSO- or NSC95397-treated cells, despite upregulation of IFNβ expression in NSC95397-treated cells when compared to DMSO-treated cells at these time points following infection. This finding suggests that NSC95397 did not act to upregulate IFNs expression by relieving the inhibition of cytoplasmic RLR signaling.

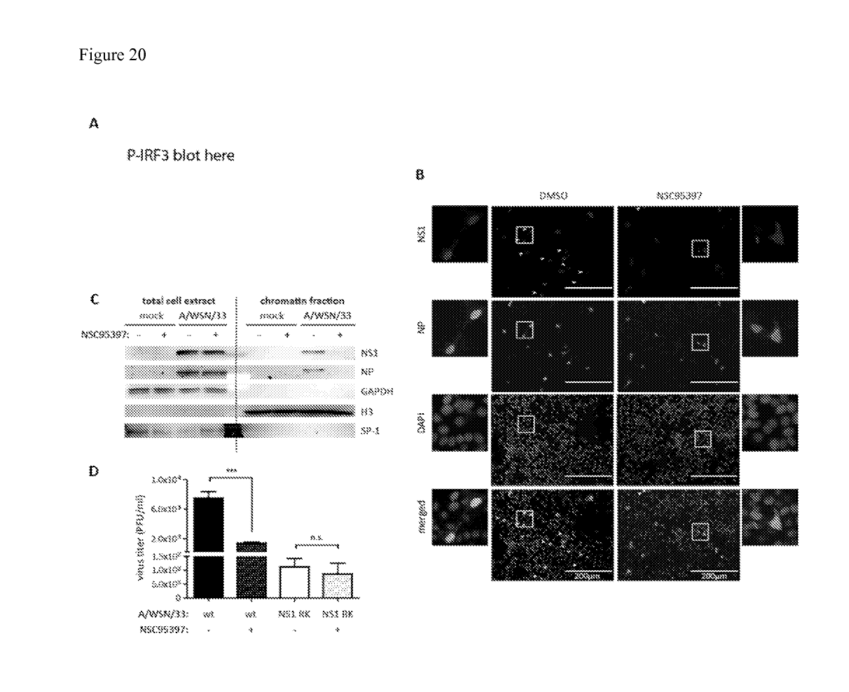

NS1 protein is also known to be differentially localized in IAV-infected cells to modulate the host response to infection by features attributed to a nuclear localization signal (NLS) and nuclear export signals (NES) (Melén et al., 2007. Journal of Virology 81:5995-6006, Greenspan et al., 1988. Journal of Virology 62:3020-3026, Li et al., 1998. Proceedings of the National Academy of Sciences 95:4864-4869). IAV expressing non-phosphorylated NS1 protein is attenuated in vitro, displaying slower growth, smaller plaque size, and delayed localization into discrete intra-nuclear foci (Hale et al., 2009. Virology 383:6-11). This is presumably linked to nuclear dot 10 (ND 10) structures and/or the site of active antiviral gene transcription as previously reported (Marazzi et al., 2012. Nature 483:428-433, Sato et al., 2003. Virology 310:29-40). Since NS1 has also been reported to block host antiviral gene expression downstream of the RLR signaling, the effect of NSC95397 on nuclear NS1 function was further evaluated. To determine if NS1 localization is modulated in the presence of NSC95397, BEAS2B cells were infected with IAV in the presence of DMSO or 2 μM NSC95397, fixed at 7 hpi, and stained for viral NP and NS1 proteins. Consistent with previous findings (Melén et al., 2007. Journal of Virology 81:5995-6006, Elton et al., 2001. Journal of Virology 75:408-419), NP and NS1 proteins were nuclear at 7 hpi in DMSO-treated cells (FIG. 20B). However, NSC95397-treated cells displayed diffused NS1 staining, but nuclear NP staining, suggesting that NS1 protein is no longer retained in the nucleus in NSC95397-treated cells. Since NS1 nuclear foci is observed in certain IAV strains (Melén et al., 2007. Journal of Virology 81:5995-6006), NS1 chromatin association was also determined using subcellular fractionation and nucleases to extract chromatin-bound proteins from an insoluble nuclear pellet. In DMSO-treated BEAS2B cells infected with A/WSN/33, NS1 and NP proteins were found to be associated with cellular chromatin (FIG. 20C). This finding is in agreement with a previous finding that NS1 is chromatin-bound to prevent transcription elongation of antiviral genes and that vRNP is found associated with cellular chromatin (Perwitasari et al., 2012. Antimicrobial agents and chemotherapy, Chase et al., 2011. PLoS Pathog 7:e1002187). In contrast, NS1 and NP proteins are not found in the chromatin fraction of infected cells treated with NSC95397. GAPDH (a cytoplasmic protein) and SP 1 (a soluble nuclear protein) were not found in the chromatin fraction, whereas histone H3 was enriched in the chromatin fraction.

To confirm that NSC95397 acts to block NS1 function, growth of recombinant influenza A/WSN/33 virus expressing defective NS1 (NS1 R38AK41A (NS1 RK)) was assessed in presence of DMSO or NSC95397 (FIG. 20D). In agreement with our previous findings, lower wild type A/WSN/33 virus titer was significantly lower at 24 hpi in cells treated with NSC95397 (p<0.001). However, growth of A/WSN/33 NS1 RK virus was not affected by NSC95397 treatment, demonstrating NSC95397 act to limit IAV replication by inhibition of NS1 action.

NSC95397 protects mice against lethal IAV infection. To assess if NSC95397 can be used to limit IAV replication in vivo, mice were treated with 2.5 mg/kg NSC95397 at 24 hours pre- or post-challenge with lethal dose (10³ PFU) of A/WSN/33 virus. Mice were monitored daily for 14-days to observe survival (FIG. 6A) and weight loss (FIG. 6B). Mice treated with NSC95397 pre- or post-infection were fully protected against lethal A/WSN/33 infection (p<0.001) and displayed less severe weight loss compared to DMSO-treated mice (p<0.001 or p<0.5, respectively). To assess lung viral burden, mice were treated with DMSO or increasing dose of NSC95397 pre- (prophylactic) or post-infection (therapeutic) and infected with sub-lethal dose (70 PFU) of A/WSN/33 (FIG. 6C). At 72 hpi, mice treated prophylactically with NSC95397 at 2.5 mg/kg (24 hours pre-infection; p<0.01) and at 5 mg/kg (two administrations of 2.5 mg/kg each at 24 and 12 hours pre-infection; p<0.001) displayed significant reduction of lung virus copy number. Additionally, 5 mg/kg NSC95397 administered therapeutically (two administration of 2.5 mg/kg dose at 12 and 24 hpi) also significantly reduced lung virus copy number (p<0.001). Suboptimal NSC95397 dose (0.5 mg/kg) administered prophylactically or therapeutically did not significantly reduce virus copy number. At sub-lethal infection dose, single 2.5 mg/kg dose of NSC95397 administered at 24 hpi also failed to reduce lung virus burden, although this particular dose was protective against lethal WSN infection. This can be attributed to partial reduction of disease which does not always correlate with viral burden. In agreement with this, therapeutic (+24 hr) treatment of NSC95397 only partially protect mice from weight loss associated with lethal infection, although this protection was still statistically significant (p<0.05). Taken together, these results demonstrate that inhibition of CDC25B function by its small molecule inhibitor NSC95397 can potentially be used as a novel IAV antiviral therapeutic strategy.

### Discussion

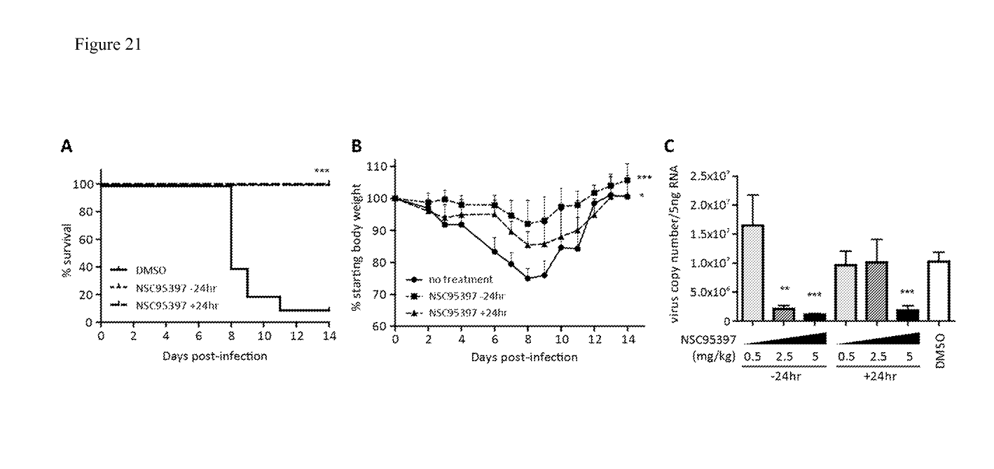

There are limited influenza drugs available, and few new drug therapies or approaches reported to control influenza virus replication (Min et al., Nat Biotechnol 28:239-240). However, as investigators have begun to harness the power of RNAi, a greater understanding of how influenza viruses co-opts host cell pathways to facilitate replication is being uncovered (Watanabe et al., Cell Host Microbe 7:427-439, Konig et al., 2010. Nature 463:813-817), and this is opening new avenues for drug targeting and repurposing for specific host cell pathways. This present study is a continuation of earlier work that identified and validated mammalian host genes in A549 type-II respiratory epithelial cells required for A/WSN/33 replication using a high throughput siRNA screening approach similar to that described by others (Brass et al., 2009. Cell 139:1243-1254, Hao et al., 2008. Nature 454:890-893, Karlas et al., 2010. Nature 463:818-822, Konig et al., 2010. Nature 463:813-817, Perwitasari et al., 2012. Antimicrobial agents and chemotherapy, Meliopoulos et al., 2012. FASEB J., 26:1372-1386, Meliopoulos et al., 2012. PloS one 7:e37169, Shapira et al., 2009. Cell 139:1255-1267, Sui et al., 2009. Virology 387:473-481). One gene in the phosphatase family, i.e. the CDC25B gene is critical for A/WSN/33 replication in A549 cells (FIG. 16). In the present study, inhibition of the CDC25B using NSC95397 was shown to prevent IAV replication in BEAS2B cells. NSC95397 (p-naphthoquinone) is a small molecule previously identified to inhibit CDC25B activity in vitro through a screen of 10,070 compounds against recombinant human CDC25B, and is the most potent CDC25 inhibitor described to date (Lazo et al., 2002. Mol Pharmacol 61:720-728, Park et al., 2007. Bioorg Med Chem Lett 17:2351-2354). CDC25B is a proto-oncogene as it facilitates mitotic entry during the cell cycle progression. Thus overexpression of CDC25B has been reported in several cancers, is associated with a poor prognosis, and its inhibition has been suggested for anti-cancer therapeutics (Kristjansdottir et al., 2004. Chem Biol 11:1043-1051, Bugler et al., 2010. Molecular Cancer 9:29, Boutros et al., 2007. Nature reviews. Cancer 7:495-507, Lyon et al., 2002. Nat Rev Drug Discov 1:961-976). NSC95397 is thought to inhibit CDC25B phosphatase activity function by covalently modifying serine residues on the active site of CDC25 and has been shown to increase levels of phosphorylated CDC25B targets such as CDK1, CDK2, and ERK (FIG. 17) (Nemoto et al., 2004. The Prostate 58:95-102, Peyregne et al., 2005. Molecular Cancer Therapeutics 4:595-602). In this study, pre-treatment of BEAS2B cells with NSC95397 effectively reduced replication of influenza A/WSN/33 and B/Florida/04/06 viruses, a representative strain of influenza A and B virus, respectively (FIG. 18). Furthermore, mice treated with NSC95397, administered prophylactically (pre-infection) or therapeutically (post-infection) were completely protected against lethal A/WSN/33 virus challenge (FIG. 21), which suggests potential use of NSC95397 as an IAV therapeutic.

Although NSC95397 was found to be effective to limit IAV infection, its mechanism of action against IAV is unclear. One possibility is that a decrease of CDC25B phosphatase activity could result in inhibition of its target, i.e. the CDK/cyclin complexes affecting influenza virus replication. Many RNA and DNA viruses depend on the host cell cycle for replication, with some like Simian virus 40 (DeCaprio et al., 1988. Cell 54:275-283) and adenovirus (Eckner et al., 1994. Genes & development 8:869-884) encoding proteins that promote cell cycle progression to support viral replication. In contrast, human immunodeficiency virus type 1 (HIV-1) encodes viral proteins that induce cell cycle arrest in G2/M phase (Goh et al., 1998. Nature medicine 4:65-71). A recent report demonstrated that upregulation of cell cycle molecules including CDC25B and CDK2 may be linked to disease severity associated with IAV infection (Parnell et al., 2011. PloS one 6:e17186). It is likely that more than one viral protein may be involved in host cell cycle modulation; however, IAV NS1 protein was recently shown to be phosphorylated by CDK1 at its threonine-215 residue (Hale et al., 2009. Virology 383:6-11). Recombinant IAV expressing non-phosphorylatable NS1 protein was attenuated in vitro, displayed slower growth, smaller plaque size, and displayed slower nuclear localization of NS1 protein (Hale et al., 2009. Virology 383:6-11).

To determine when and where CDC25B is involved in IAV replication, NSC95397 was added to BEAS2B cells before or after IAV infection. NSC95397 was found to be effective only when added prior to 6 hpi (FIG. 19), suggesting CDC25B has a role to support IAV replication mid-cycle. Regarding the IAV replication cycle, vRNP has been shown to be imported into the nucleus between 1.5 to 2 h post-virus binding and uncoating (Samji, 2009. The Yale journal of biology and medicine 82:153-159, Boulo et al., 2007. Virus Res 124:12-21). Once in the nucleus, (+) sense viral mRNA is synthesized using the incoming (−) sense viral genomic RNA (vRNA) for subsequent viral protein translation in the cytoplasm (Neumann et al., 2004. Current topics in microbiology and immunology 283:121-143, Vreede et al., 2007. J Virol 81:2196-2204). Additionally, (+) sense viral cRNA is also synthesized from vRNA as templates for nascent vRNA, followed by vRNP export to the cytoplasm at approximately 8 hpi, for packaging and release of new viral progenies to complete viral replication cycle which is approximately 12 hpi (Nayak et al., 2004. Virus Res 106:147-165, Samji, 2009. The Yale journal of biology and medicine 82:153-159, Paterson et al., 2012. PLoS Pathog 8:e1003019). Based on this outline of IAV replication, the effect of NSC95397 to alter the abundance of specific viral RNA species was evaluated. NSC95397 was found to specifically reduce (+) sense viral RNAs, i.e. mRNA and cRNA, while upregulating expression of host type-I and type-III IFNs (FIG. 19). Various reports have demonstrated influenza virus NS1 protein's action to block the RIG-1-like receptors (RLR) signaling upstream of IRF3 activation to suppress host's IFN expression (Hale et al., 2008. Journal of General Virology 89:2359-2376, Mibayashi et al., 2007. J Virol 81:514-524, Opitz et al., 2007. Cellular microbiology 9:930-938, Guo et al., 2007. American journal of respiratory cell and molecular biology 36:263-269). Interestingly, NSC95397 did not result in increased IRF3 phosphorylation (FIG. 20). This suggests that NSC95397 did not act to upregulate IFN expression by inhibiting NS1 action in the cytoplasm to block intracellular pattern recognition receptor signaling (i.e., RLR signaling).

Due to diverse roles of NS1 that include modulating host processes, and the evidence that NS1 phosphorylation by CDC25B target CDK1 was shown to modulate its nuclear localization, NSC95397 activity on NS1 function was evaluated. One interesting finding is the demonstration that NS1 is chromatin-bound to prevent transcription elongation of antiviral genes (Marazzi et al., 2012. Nature 483:428-433, Chase et al., 2011. PLoS Pathog 7:e1002187). In this study, NSC95397 treatment was found to modulate nuclear localization of NS1 protein and its association with cellular chromatin (FIG. 20). Based on this finding and previously reported functions, we postulated CDC25B promotes IAV replication by activating CDK1 and ERK kinases, a step that is blocked by CDC25B inhibitor NSC95397, to phosphorylate NS1 at the threonine-215 residue which ultimately resulted in repression of host antiviral gene expression, such as type-I and -III IFNs.

Taken together, this study show that a better understanding of the host genes required for IAV replication can provide critical information about host cell pathways co-opted by influenza virus, and this in turn can be used to repurpose or reposition existing drugs to inhibit functions of these host factors and limit virus replication. The studies performed here utilized BEAS2B cells that are biosimilar to normal bronchial epithelium and corroborated findings in a mouse model. Importantly, this study demonstrates that whole-genome siRNA screens (such as siGENOME screen) can be used to identify host genes critical for IAV replication, which can then be translated to other cell culture systems as well as in vivo murine studies, features that should hasten novel drug anti-viral discovery for IAV.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method for treating a subject comprising:
administering to a subject an effective amount of a composition comprising 4-(dipropylsulfamoyl)benzoic acid, wherein the subject has, or is at risk of having, an influenza virus infection, wherein the subject comprises no greater than a detectable level of oseltamivir carboxylate.

2. A method for treating a subject comprising:
administering to a subject an effective amount of a composition comprising 4-(dipropylsulfamoyl)benzoic acid, wherein the subject has, or is at risk of having, a virus infection, wherein the virus infection is not an influenza virus infection

3. A method for treating a subject comprising:
administering to a subject an effective amount of a composition comprising an inhibitor of CDC25B phosphatase, wherein the subject has, or is at risk of having, a virus infection.

4. (canceled)

5. The method of claim 2 further comprising administering to the subject a composition comprising an inhibitor of CDC25B phosphatase, an inhibitor of CamK2B, an inhibitor of ICAM-1, or a combination thereof.

6. The method of claim 3 further comprising administering to the subject a composition comprising an inhibitor of CamK2B, an inhibitor of ICAM-1,4-(dipropylsulfamoyl)benzoic acid, or a combination thereof.

7. (canceled)

8. The method of claim 6 wherein one or more hydrogen-bearing carbon atoms in the 4-(dipropylsulfamoyl)benzoic acid is substituted, wherein each substituent is selected from a halogen, a nitrile, a hydroxy, an alkoxy (OR), a nitrate, a nitrite, a sulfate (O—SO₃R), an amino (NR₂), a nitro, a sulfonate (SO₂OR), or a C1-C10 organic group, wherein each R is independently a hydrogen or an organic group.

9. The method of claim 3 wherein the inhibitor of CDC25B phosphatase is selected from NSC95397, NSC115447, NSC135880, NSC139049, or NSC672121.

10. The method of claim 9 wherein one or more hydrogen-bearing carbon atoms in the NSC95397, NSC115447, NSC135880, NSC139049, or NSC672121 is substituted, wherein each substituent is selected from a halogen, a nitrile, a hydroxy, an alkoxy (OR), a nitrate, a nitrite, a sulfate (O—SO₃R), an amino (NR₂), a nitro, a sulfonate (SO₂OR), or a C1-C10 organic group, wherein each R is independently a hydrogen or an organic group.

11. The method of claim 6 wherein the inhibitor of ICAM-1 is 4-[(4-Methyl phenyl)thio]thieno[2,3-c]pyridine-2-carboxamide.

12. The method of claim 11 wherein one or more hydrogen-bearing carbon atoms in the 4-[(4-Methyl phenyl)thio]thieno[2,3-c]pyridine-2-carboxamide is substituted, wherein each substituent is selected from a halogen, a nitrile, a hydroxy, an alkoxy (OR), a nitrate, a nitrite, a sulfate (O—SO₃R), an amino (NR₂), a nitro, a sulfonate (SO₂OR), or a C1-C10 organic group, wherein each R is independently a hydrogen or an organic group.

13. The method of claim 6 wherein the inhibitor of CamK2B is KN-62, KN-93, arcyriaflavin A or SEQ ID NO:10.

14. The method of claim 13 wherein one or more hydrogen-bearing carbon atoms in the KN-62, KN-93, or arcyriaflavin A is substituted, wherein each substituent is selected from a halogen, a nitrile, a hydroxy, an alkoxy (OR), a nitrate, a nitrite, a sulfate (O—SO₃R), an amino (NR₂), a nitro, a sulfonate (SO₂OR), or a C1-C10 organic group, wherein each R is independently a hydrogen or an organic group.

15. The method of claim 3 wherein the virus infection is a virus infection of the respiratory tract.

16. The method of claim 15 wherein the virus infection comprises an influenza virus.

17. The method of claim 16 wherein the influenza virus is influenza virus A.

18. The method of claim 16 wherein the influenza virus is influenza virus B.

19-22. (canceled)
